(19) United States
(12) Patent Application Publication
(10) Pub. No.: US 2003/0100769 A1
(43) Pub. Date: May 29, 2003

(54) Cyclic amidine compounds

(76) Inventors: Masahiro Imoto, Tatebayashi-shi (JP); Tatsuya Iwanami, Ashikaga-shi (JP); Minako Akabane, Ibaraki-shi (JP); Yoshihiro Tani, Ibaraki-shi (JP)

Correspondence Address: CROWELL & MORING LLP, INTELLECTUAL PROPERTY GROUP, P.O. BOX 14300, WASHINGTON, DC, 20044-4300, US

(21) Appl. No.: 10/009,477

(22) PCT Filed: Apr. 20, 2001
(86) PCT No.: PCT/JP01/03378

(30) Foreign Application Priority Data
Apr. 21, 2000 (JP) 2000-120976

(51) Int. Cl. C07D 233/04; C07D 233/54

(52) U.S. Cl. 548/311.1; 544/182; 548/335.1; 548/347.1

(57) ABSTRACT

There is provided cyclic amidine compounds of the following formula (I):

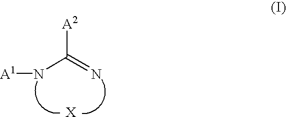

wherein:

A¹ and A² are hydrogen atom, optionally substituted alkyl group; optionally substituted aryl group; or optionally substituted heterocyclic group; and

X is —C(R¹,R²)—C(R³,R⁴)—, —C(R⁵)═C(R⁶)—, —C(R⁷,R⁸)—C(R⁹,R¹⁰)—C(R¹¹,R¹²)—, or —C(R¹³,R¹⁴)—C(R¹⁵,R¹⁶)—NH— (wherein, R¹, R², R³, R⁴, R⁵, R⁶, R⁷, R⁸, R⁹, R¹⁰, R¹¹, R¹², R¹³, R¹⁴, R¹⁵ and R¹⁶ are hydrogen atom; halogen atom; optionally substituted alkyl group; optionally substituted aryl group; or optionally substituted heterocyclic group;

or pharmaceutically acceptable salts thereof.

These compounds have good affinity for α4β2 nicotinic acetylcholine receptors and activate the same to thereby exert a preventive or therapeutic effect on cerebral dysfunction.

### TECHNICAL FIELD

The present invention relates to compounds showing affinity for nicotinic acetylcholine receptors and activating the same. The compounds of the present invention are useful for preventing or treating of neurodegenerative diseases such as Alzheimer's disease and Parkinson's disease, dementia such as cerebrovascular dementia, motor ataxia such as Tourette's syndrome, neurosis during chronic cerebral infarction stage, neuropathy and mental disorder such as anxiety and schizophrenia and cerebral dysfunction caused by cerebral injury.

### BACKGROUND ART

It has been widely known that nicotine exerts a wide variety of pharmacological effects. These include, for example, cholinergic nervous activation as the effect on central nervous systems such as facilitation of acetylcholine release [De sarno P. & Giacobini E., J. Neurosci. Res., 22, 194-200 (1984)], and further, activation effect on monoaminergic nervous systems [Levin E. D. & Simon B. B., Psychopharmacology, 138, 217-230 (1998)].

It has been also reported that nicotine possesses lots of very useful cerebral function improving effects such as increasing cerebral blood flow and glucose uptake rate in brain [Decker M. W. et al., Life Sci., 56, 545-570 (1995)].

It has been further reported that nicotine inhibits amyloid formation of β-peptides which is believed to be the cause of neuronal cell death during Alzheimer's disease [Salomon A. R. et al., Biochemistry, 35, 13568-13578 (1996)], and have cell protective effects on neuronal cell death induced by β-amyloid (Aβ) [Kihara T. et al., Ann. Neurol., 42, 156-163 (1997)]. Recent studies suggest the possibility of nicotine being a remedy for the inflammatory colitis [Sandborn W. J. et al., Ann. Intern. Med., 126, 364 (1997)].

On the other hand, it is acknowledged that in the patients of Alzheimer's disease, the degeneration of acetylcholinergic neurons known to be one of the important nervous systems responsible for cognition such as attention, learning, memory and recognition, is altered and thus nicotinic acetylcholine receptors in the cerebral cortex and hippocampus are drastically decreased [Nordberg A. et al., J. Neurosci. Res., 31, 103-111 (1992)].

It is reported the possibility of the useful treatment for Alzheimer's disease by activating nicotinic acetylcholine receptors to be recovered the acetylcholine nervous systems mechanism by agonists or modulators of nicotinic acetylcholine receptors [Newhouse P. A. et al., Psychopharmacology, 95, 171-175 (1988)].

The nicotinic acetylcholine receptors belong to the ion channel neurotransmitter receptors composed of five subunits. That is, agonists such as acetylcholine, nicotine and the like are bound to receptors to activate and open the channels thereof, thus causing the influx of cationic ion such as sodium ion from extracellular to result the cell excitation [Galzi J. L. & Changeux J. P., Neuropharmacology, 34, 563-582 (1995)]. The aforementioned agonists such as acetylcholine, nicotine and the like show its effect by binding to the specific site existing in α subunit so-called agonist binding site.

It is known, on the other hand, that compounds such as galantamine and so on which activate cells by potentiating the effects of acetylcholine, have no agonist effect at nicotinic acetylcholine receptors directly. These compounds show their effects through allosteric site which is clearly different from the agonist binding sites [Schrattenholz A. et al., Mol. Pharmacol., 49, 1-6 (1996)].

Mentioned above, compounds capable to activate nicotinic acetylcholine receptors indirectly are called modulators and it is expected to be the practical medicines for treatment of the various neurological diseases [Lin N. -H & Meyer M. D., Exp. Opin. Thr. Patents, 8, 991-1015 (1998)].

The terms “agonists” and “modulators” are used in these definitions in the present specification.

The nicotinic acetylcholine receptors are believed to participate not only in Alzheimer's disease, but also in neurodegenerative diseases such as Parkinson's disease, and many of the neuroses and psychoses such as dementia, anxiety, schizophrenia and so on [Barrantes F. J., in The Nicotic Acetylcholine Receptor, ed. Barrantes F. J., Springer, 1997, p175-212; Lena C. & Changeux J. -P., J. Physiol. (Paris), 92, 63-74 (1998)].

Especially, since it is known that cerebral blood flow of the patients suffering from cerebrovascular dementia caused by cerebral infarction is decreased [Takagi Shigeharu, Gendai Iryo, 28, 1157-1160 (1996); Tachibana H. et al., J. Gerontol., 39, 415-423 (1984)], there seems to be the possibility of agonists of nicotinic acetylcholine receptors or the modulators possessing cerebral blood flow increasing effect to be applied to the medicines in this area of treatment. Furthermore, recent study revealed that agonists of nicotinic acetylcholine receptors and the modulators thereof show analgesic activities [Bannon A. W. et al., Science, 279, 77-81 (1998)].

Nicotine itself surely affects as the agonist of nicotinic acetylcholine receptors. For example, after administration of nicotine to the patients of Alzheimer's disease, the recoveries of their attention or the short-term memory were observed, and also the symptoms of their disease were improved [Newhouse P. A. et al., Drugs & Aging, 11, 206-228 (1997)]. Nevertheless, nicotine also possesses disadvantages such as widely recognized addiction, as well as low bioavailability and severe side effects to the cardiovascular systems.

Therefore, there have been great expectation to develop nicotinic acetylcholine receptors agonists or modulators as medicines in place of nicotine which has no addiction, high bioavailability, and less side effects on cardiovascular systems [Maelicke A. & Albuquerque E. X., Drug Discovery Today, 1, 53-59 (1996); Holladay M. W. et al., J. Med. Chem., 40, 4169-4194 (1997)].

There are some subtypes known as the nicotinic acetylcholine receptors [Shacka J. J. & Robinson S. E. T. , Med. Chem. Res., 1996, 444-464], and mainly α4β2 subtype receptors exist in central nervous systems. Furthermore, there exist alply α1β1γδ (or α1β1εδ) subtype receptors in the neuromuscular junction of motor neurons, and α3β4 subtype receptors in ganglion of autonomic nervous systems and adrenal.

The activation of the cholinergic nervous systems and increasing effect of cerebral blood flow are believed to occur though α4β2 subtype receptors in central nervous systems, and above mentioned effects of nicotine on cardiovascular system are induced by affecting receptor subtypes exist in peripheral nervous system.

Therefore, it may be extremely useful as medicines having no side effects to develop compounds which have no affinity at α1β1γδ subtype nor α3β4 subtype receptors, but selectively affects α4β2 subtype receptors.

In these circumstances, there have been many proposals to develop selective agonists or modulators at nicotinic acetylcholine receptors of central nervous system as practical medicines. These include, for example, the compound such as ABT-418 [Arneric S. P. et al., J. Pharmacol. Exp. Ther., 270, 310-318 (1994); Decker M. W. et al., J. Pharmacol. Exp. Ther., 270, 319-328 (1994)], ABT-089 [Sullivan J. P. et al., J. Pharmacol. Exp. Ther., 283, 235-246 (1997); Decker M. W. et al., J. Pharmacol. Exp. Ther., 283, 247-258 (1997)], GTS-21 [Arendash G. W. et al., Brain Res., 674, 252-259 (1995); Briggs C. A. et al., Pharmacol. Biochem. Behav., 57, 231-241 (1997)], RJR-2403 [Bencherif M. et al., J. Pharmacol. Exp. Ther., 279, 1413-1421 (1996); Lippiello P. M. et al., J. Pharmcol. Exp. Ther., 279, 1422-1429 (1996)], SIB-1508Y [Cosford N. D. P. et al., J. Med. Chem., 39, 3235-3237 (1996); Lloyd. G. K. et al., Life Sci., 62, 1601-1606 (1995)], SIB-1553A [Lloyd. G. K. et al., Life Sci., 62, 1601-1606 (1995)] and so on.

In European Patent Publication EP679397-A2, substituted amine derivatives represented by the following formula were proposed for the medicines for prevention and treatment of cerebral dysfunction.

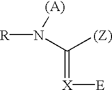

in which,

R represents hydrogen, optionally substituted acyl, alkyl, aryl, aralkyl, heteroaryl or heteroarylalkyl radicals;

A represents a monofunctional group of the hydrogen, acyl, alkyl or aryl series or represents a bifunctional group which is linked to the radical Z;

E represents an electron-withdrawing radical;

X represents the —CH═ or ═N— radicals, it being possible for the —CH═ radical to be linked to the Z radical instead of an H atom;

Z represents a monofunctional group of the alkyl, —O—R, —S—R or —NR₂ series or represents a bifunctional group which is linked to the A radical or the X radical.

However, the structure of the compounds disclosed in said patent publication is clearly different from that of the compounds disclosed by the present patent application, and there is no description in the above-mentioned patent publication that these compounds can selectively activate α4β2 nicotinic acetylcholine receptors.

On the other hand, it is confirmed that “imidacloprid”, as a pesticide, electrophysiologically affects as partial agonist at nicotinic acetylcholine receptors of PC12 cell [Nagata K. et al., J. Pharmacol. Exp. Ther., 285, 731-738 (1998)], and imidacloprid itself or its metabolites and their analogues possess affinity to the nicotinic acetylcholine receptors in mouse brain [Lee Chao S. & Casida E., Pestic. Biochem. Physiol., 58, 77-88 (1997); Tomizawa T. & Casida J. E., J. Pharmacol., 127, 115-122 (1999); Latli B. et al., J. Med. Chem., 42, 2227-2234 (1999)], however, there is no report of the imidacloprid derivatives selectively activating α4β2 nicotinic acetylcholine receptors. Furthermore, the structure of the imidacloprid itself or its metabolites and their analogues is clearly different from that of the compounds disclosed by the present patent application.

Japanese Laid-open Patent Publication Number Hei 10-226684 disclosed [N-(pyridinylmethyl)heterocyclic]ylideneamine compounds represented by the following formula, pharmaceutically acceptable salts and prodrugs thereof.

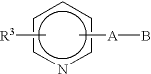

in which,

A represents the —CH(R)—;

R³ represents a hydrogen atom or an optionally substituted C₁-C₆ alkyl; and

B represents the group of the following formula:

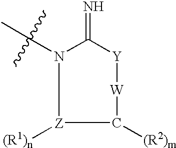

It is disclosed that these compounds possess weak affinity to nicotinic receptors; however, there is no description that these compounds have selective activating effect at α4β2 nicotinic acetylcholine receptors of central nervous systems and act as agonists or modulators of nicotinic acetylcholine receptors. Furthermore, the structure of these compounds is clearly different from that of the compounds disclosed by the present invention.

As mentioned above, there had been many attempts to develop agonists or modulators selectively activating α4β2 nicotinic acetylcholine receptors of central nervous systems via oral administration, but none were satisfactory.

### DISCLOSURE OF THE INVENTION

Therefore, the present invention provides therapeutic or preventing agents for treatment of diseases which may be prevented or cured by activating nicotinic acetylcholine receptors, having the capabilities of binding selectively with α4β2 nicotinic acetylcholine receptor of central nervous systems, and having no undesirable side effects in cardiovascular systems such as hypertension or tachycardia.

More specifically, the present invention provides medicaments for preventing or treating various diseases, which may be prevented or cured by activating nicotinic acetylcholine receptors, such as dementia, senile dementia, presenile dementia, Alzheimer's disease, Parkinson's disease, cerebrovascular dementia, AIDS-related dementia, dementia in Down's syndrome, Tourette's syndrome, neurosis during chronic cerebral infarction stage, cerebral dysfunction caused by cerebral injury, anxiety, schizophrenia, depression, Huntington's disease, pain and so on.

Through extensive investigations of researching compounds having the capabilities of binding selectively with α4β2 nicotinic acetylcholine receptors of central nervous systems, the present inventors discovered that the compounds represented by the formula (I) mentioned below and pharmaceutically acceptable salts thereof possess high affinity for nicotinic acetylcholine receptors in central nervous systems, and activate said receptors as agonists or modulators.

Accordingly, as one aspect of the present invention, it is provided cyclic amidine compounds represented by the following formula (I):

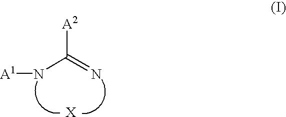

wherein:

A¹ and A² are hydrogen atom, optionally substituted alkyl group; optionally substituted aryl group; or optionally substituted heterocyclic group; and

X is —C(R¹,R²)—C(R³,R⁴)—, —C(R⁵)═C(R⁶)—, —C(R⁷,R⁸)—C(R⁹,R¹⁰)—C(R¹¹,R¹²)—, or —C(R¹³,R¹⁴)—C(R¹⁵, R¹⁶)—NH— (wherein, R¹, R², R³, R⁴, R⁵, R⁶, R⁷, R⁸, R⁹, R¹⁰, R¹¹, R¹², R¹³, R¹⁴, R¹⁵ and R¹⁶ are hydrogen atom; halogen atom; optionally substituted alkyl group; optionally substituted aryl group; or optionally substituted heterocyclic group; or pharmaceutically acceptable salts thereof.

Still another aspect of the present invention, it is provided activator agents for α4β2 nicotinic acetylcholine receptors containing cyclic amidine compounds of the formula (I) or pharmaceutically acceptable salt thereof as active ingredients.

As still further aspect of the present invention, it is provided that the use of cyclic amidine compounds of the formula (I) or pharmaceutically acceptable salt thereof for treating or preventing of cerebral circulation disease, neurodegenerative disease and the like.

### BEST MODE FOR CARRYING OUT THE INVENTION

Examples of the pharmaceutically acceptable salt include an inorganic acid salt such as hydrochloric acid salt, hydrobromic acid salt, sulfuric acid salt, phosphoric acid salt and the like, and an organic acid salt such as fumaric acid salt, maleic acid salt, oxalic acid salt, citric acid salt, tartaric acid salt, malic acid salt, lactic acid salt, succinic acid salt, benzoic acid salt, methanesulfonic acid salt, p-toluenesulfonic acid salt and the like.

The groups represented by “A¹” and “A²” in the compound of formula (I) are hydrogen atom, optionally substituted alkyl group, optionally substituted aryl group or optionally substituted heterocyclic group, and preferable examples of said optionally substituted alkyl group include methyl, ethyl, propyl, isopropyl, butyl, sec-butyl, tert-butyl and the like.

Suitable substituent of substituted alkyl group may include optionally substituted aryl group or optionally substituted heterocyclic group, and therefore, examples of said substituted alkyl group include benzyl, (2-pyridyl)methyl, (3-pyridyl)methyl, (2-chloro-3-pyridyl)methyl, (6-chloro-3-pyridyl)-methyl, (6-fluoro-3-pyridyl)methyl, (5-bromo-3-pyridyl)methyl, (2,6-dichloro-3-pyridyl)methyl, (5,6-dichloro-3-pyridyl)methyl, (2,6-dichloro-3-pyridyl)methyl, (6-methyl-3-pyridyl)methyl, (6-ethoxy-3-pyridyl)methyl, (5-pyrimidyl)methyl, (3-quinolyl)-methyl, (3-furanyl)methyl, (tetrahydro-3-furanyl)-methyl, (3-thienyl)-methyl, (3,5-dimethylisoxazolyl)methyl, 1-(6-chloro-3-pyridyl)-ethyl, 2-(6-chloro-3-pyridyl)ethyl and the like.

The preferable examples of aryl group of said optionally substituted aryl group represented by “A¹” and “A²” may include phenyl, naphthyl and the like. Suitable substituent of substituted aryl group may include C₁-C₄ lower alkyl group, hydroxyl group, amino group, halogen atom and the like, and therefore, examples of said substituted aryl group include methylphenyl, hydroxyphenyl, aminophenyl, chlorophenyl, dichlorophenyl and the like.

The term “heterocyclic group” represented by “A¹” and “A²” may be 5 or 6 membered heterocyclic group or condensed heterocyclic group thereof containing the same or different 1 to 3 hetero atom(s) such as sulfur, nitrogen, oxygen atom(s), and examples include thiophene, furan, pyran, pyrrole, pyrazole, pyridine, pyrimidine, pyrazine, pyridazine, imidazole, oxazole, isoxazole, thiazole, isothiazole, quinoline, isoquinoline, indole, azaindole, tetrahydropyrimidine and the like.

Suitable substituent of substituted heterocyclic group may include C₁-C₄ lower alkyl, halogen atom and the like, and therefore, examples of said substituted heterocyclic group may be 2-methylpyridine, 6-methylpyridine, 2-chloropyridine, 2-fluoropyridine, 2-bromopyridine, 3-bromopyridine, 2,3-dichloropyridine, 2-chloropyrimidine, 2-chlorothiazole, 3,5-dimethylisoxazole and the like.

The group represented by “X” is the partial component of the bond as following;

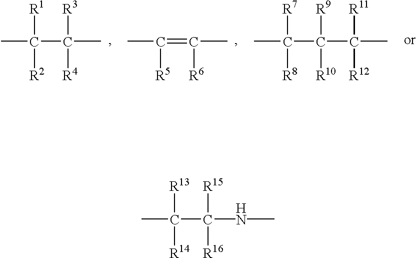

wherein, R¹ to R¹⁶ are hydrogen atom; halogen atom; optionally substituted alkyl group; optionally substituted aryl group; or optionally substituted heterocyclic group.

The term “halogen atom” represented by R¹, R², R³, R⁴, R⁵, R⁶, R⁷, R⁸, R⁹, R¹⁰, R¹¹, R¹², R¹³, R¹⁴, R¹⁵ and R¹⁶ may include fluorine, chlorine, bromine and iodine.

The term “optionally substituted alkyl group” may include methyl, ethyl, propyl, isopropyl, butyl, sec-butyl, tert-butyl and the like.

Suitable substituent of substituted alkyl group may include optionally substituted aryl group or optionally substituted heterocyclic group, and therefore, examples of said substituted alkyl group include benzyl, (2-pyridyl)methyl, (3-pyridyl)methyl, (2-chloro-3-pyridyl)methyl, (6-chloro-3-pyridyl)-methyl, (6-fluoro-3-pyridyl)methyl, (5-bromo-3-pyridyl)methyl, (2,6-dichloro-3-pyridyl)methyl, (5,6-dichloro-3-pyridyl)methyl, (2,6-dichloro-3-pyridyl)methyl, (6-methyl-3-pyridyl)methyl, (6-ethoxy-3-pyridyl)methyl, (5-pyrimidyl)methyl, (3-quinolyl)methyl, (3-furanyl)methyl, (tetrahydro-3-furanyl)methyl, (3-thienyl)-methyl, (3,5-dimethylisoxazolyl)methyl, 1-(6-chloro-3-pyridyl)-ethyl, 2-(6-chloro-3-pyridyl)ethyl and the like.

The term “optionally substituted aryl group” for the groups R¹ to R¹⁶ may be non-substituted phenyl group or phenyl group which is substituted by halogen atom, or C₁-C₄ lower alkyl such as methyl, ethyl and the like, and therefore, examples of substituted phenyl group may include methylphenyl, chlorophenyl, dichlorophenyl and the like.

The term “heterocyclic group” for the groups R¹ to R¹⁶ may be 5 or 6 membered heterocyclic group containing the same or different 1 to 3 hetero atom(s) such as sulfur, nitrogen, oxygen atom(s), and examples include thiophene, furan, pyran, pyrrole, pyrazole, pyridine, pyrimidine, pyrazine, pyridazine, imidazole, oxazole, isoxazole, thiazole, isothiazole, quinoline, isoquinoline, tetrahydropyrimidine and the like.

Suitable substituent of substituted heterocyclic group may include C₁-C₄ lower alkyl, halogen atom and the like, and therefore, examples of said substituted heterocyclic group may be 2-methylpyridine, 3-methylpyridine, 2-chloropyridine, 2-fluoropyridine, 2-bromopyridine, 3-bromopyridine, 2,3-dichloropyridine, 4-chloropyrimidine, 2-chlorothiazole, 3-methylisoxazole and the like.

The following are examples of cyclic amidine compounds of the formula (I).

Compound 1: 2-(6-chloro-3-pyridyl)-2-imidazoline;

Compound 2: 2-(6-chloro-3-pyridyl)-1,4,5,6-tetrahydropyrimidine;

Compound 3: 2-(6-chloro-3-pyridyl)-1-methyl-2-imidazoline;

Compound 4: 2-(6-chloro-3-pyridyl)-1-methyl-1,4,5,6-tetrahydropyrimidine;

Compound 5: 1-(6-chloro-3-pyridyl)methylimidazole;

Compound 6: 2-(6-chloro-3-pyridyl)imidazole;

Compound 7: 2-(6-chloro-3-pyridyl)methyl-2-imidazoline;

Compound 8: 2-(6-chloro-3-pyridyl)methyl-1,4,5,6-tetrahydropyrimidine;

Compound 9: 2-(6-chloro-3-pyridyl)methyl-1-methyl-2-imidazoline;

Compound 10: 2-(6-chloro-3-pyridyl)methyl-1-methyl-1,4,5,6-tetrahydropyrimidine;

Compound 11: 1-(6-chloro-3-pyridyl)methyl-2-methyl-2-imidazoline;

Compound 12: 1-(6-chloro-3-pyridyl)methyl-4,4-dimethyl-2-imidazoline;

Compound 13: 2-(tetrahydrofuran-3-yl)-1,4,5,6-tetrahydropyrimidine;

Compound 14: 2-(tetrahydrofuran-3-yl)-2-imidazoline;

Compound 15: 2-(tetrahydrofuran-3-yl)methyl-1,4,5,6-tetrahydropyrimidine;

Compound 16: 2-(5-bromo-3-pyridyl)methyl-1,4,5,6-tetrahydropyrimidine;

Compound 17: 2-(5-bromo-3-pyridyl)methyl-2-imidazoline;

Compound 18: 2-(3-pyridyl)methyl-1,4,5,6-tetrahydropyrimidine;

Compound 19: 2-(3-pyridyl)methyl-2-imidazoline;

Compound 20: 2-(3-aminophenyl)-1,4,5,6-tetrahydropyrimidine;

Compound 21: 2-(3-quinolyl)methyl-1,4,5,6-tetrahydropyrimidine;

Compound 22: 2-(2-chloro-5-thiazolyl)-1,4,5,6-tetrahydropyrimidine;

Compound 23: 2-(3-quinolyl)methyl-2-imidazoline;

Compound 24: 2-(2-chloro-5-thiazolyl)-2-imidazoline;

Compound 25: 2-(3-quinolyl)-1,4,5,6-tetrahydropyrimidine;

Compound 26: 2-(3-furanyl)methyl-2-imidazoline;

Compound 27: 1-(6-chloro-3-pyridyl)methyl-1,4,5,6-tetrahydropyrimidine;

Compound 28: 2-(3,5-dimethyl-4-isoxazolyl)methyl-1,4,5,6-tetrahydropyrimidine;

Compound 29: 2-(3,5-dimethyl-4-isoxazolyl)methyl-2-imidazoline;

Compound 30; 2-(3-thienyl)methyl-1,4,5, 6-tetrahydropyrimidine;

Compound 31: 2-(3-thienyl)methyl-2-imidazoline;

Compound 32: 2-methyl-5-(3-pyridyl)-2-imidazoline;

Compound 33: 5-(3-pyridyl)-2-imidazoline;

Compound 34: 1,2-bis[(6-chloro-3-pyridyl)methyl]-1,4,5,6-tetrahydropyrimidine;

Compound 35: 1-(6-chloro-3-pyridyl)methyl-2-(3-pyridyl)-2-imidazoline;

Compound 36: 2-(5,6-dichloro-3-pyridyl)methyl-1,4,5,6-tetrahydropyrimidine;

Compound 37: 2-(6-chloro-3-pyridyl)methyl-5-phenyl-1,4,5,6-tetrahydropyrimidine;

Compound 38: 2-(4-pyridyl)methyl-1,4,5,6-tetrahydropyrimidine;

Compound 39: 2-(2-chloro-3-pyridyl)methyl-1,4,5,6-tetrahydropyrimidine;

Compound 40: 2-(2,6-dichloro-3-pyridyl)methyl-1,4,5,6-tetrahydropyrimidine;

Compound 41: 2-[2-(6-chloro-3-pyridyl)ethyl]-1,4,5,6-tetrahydropyrimidine;

Compound 42: 2-[2-(6-chloro-3-pyridyl)ethyl]-2-imidazoline;

Compound 43: 2-(6-methyl-3-pyridyl)methyl-1,4,5,6-tetrahydropyrimidine;

Compound 44: 1,2-bis[(6-chloro-3-pyridyl)methyl]-2-imidazoline;

Compound 45: 2-(6-methyl-3-pyridyl)methyl-2-imidazoline;

Compound 46: 2-(6-ethoxy-3-pyridyl)methyl-1,4,5,6-tetrahydropyrimidine;

Compound 47: 2-(6-ethoxy-3-pyridyl)methyl-2-imidazoline;

Compound 48: 2-(6-fluoro-3-pyridyl)methyl-1,4,5,6-tetrahydropyrimidine;

Compound 49: 2-(5,6-dichloro-3-pyridyl)methyl-2-imidazoline;

Compound 50: 2-(6-chloro-3-pyridyl)methyl-5,5-dimethyl-1,4,5,6-tetrahydropyrimidine;

Compound 51: 2-(2-pyridyl)methyl-1,4,5,6-tetrahydropyrimidine;

Compound 52: 1-(5,6-dichloro-3-pyridyl)methyl-1,4,5,6-tetrahydropyrimidine;

Compound 53: 2-(5,6-dichloro-3-pyridyl)methyl-1-methyl-2-imidazoline;

Compound 54: 2-(6-chloro-3-pyridyl)methyl-4-methyl-1,4,5,6-tetrahydropyrimidine;

Compound 55: 1-[2-(6-chloro-3-pyridyl)ethyl]-1,4,5,6-tetrahydropyrimidine;

Compound 56: 1-(3-pyridazinyl)methyl-1,4,5,6-tetrahydropyrimidine;

Compound 57: 1-(6-methyl-3-pyridyl)methyl-1,4,5,6-tetrahydropyrimidine;

Compound 58: 1-(3-pyridyl)methyl-1,4,5,6-tetrahydropyrimidine;

Compound 59: 3-(6-chloro-3-pyridyl)methyl-1,4,5,6-tetrahydro-1,2,4-triazine;

Compound 60: 2-[1-(6-chloro-3-pyridyl)ethyl]-1,4,5,6-tetrahydropyrimidine;

Compound 61: 1-(2-chloro-5-thiazolyl)methyl-1,4,5,6-tetrahydropyrimidine;

Compound 62: 1-[2-(6-chloro-3-pyridyl)ethyl]-2-methyl-2-imidazoline;

Compound 63: 1-[2-(6-chloro-3-pyridyl)ethyl]-4,4-dimethyl-2-imidazoline;

Compound 64: 2-(2-chloro-5-thiazolyl)methyl-1,4,5,6-tetrahydropyrimidine;

Compound 65: 2-(2-chloro-5-thiazolyl)methyl-2-imidazoline;

Compound 66: 2-(5-pyrimidyl)methyl-1,4,5,6-tetrahydropyrimidine;

Compound 67: 2-(5-pyrimidyl)methyl-2-imidazoline;

Compound 68: 2-(5-methyl-3-pyridyl)methyl-1,4,5,6-tetrahydropyrimidine.

The cyclic amidine compounds represented by the formula (I) of the present invention can be prepared in accordance with the various synthetic processes such as following Process 1 to 3.

In the following reaction schemes, the groups A¹, A² and X have the same meanings mentioned above.

Process 1:

In accordance with the following reaction scheme, the compound (I) of the present invention can be obtained by the condensation reaction of the compound of the formula (II) with the compound of the formula (III).

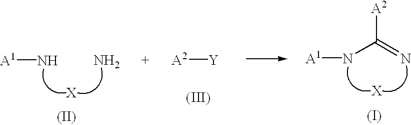

wherein, “Y” is —COOQ¹, —CONQ^{′2}Q³, —C(OQ⁴)₃, —C(OQ⁵)═NH or —CN (in which Q¹, Q², Q³, Q⁴ and Q⁵ are C₁-C₄ lower alkyl); that is, the compound (III) represented by “A²-Y” is carboxylic acid derivative such as ester, amide, orthoester, iminoether or nitrile.

The compounds (II) and (III) to be used in this reaction can be commercially available or can be easily prepared from known compounds by using common methods.

The reaction of the compound (II) with the compound (III) to obtain the compound (I) can usually be carried out without solvent or in an appropriate solvent such as hydrocarbon solvent, alcohol solvent and ether solvent or the mixture thereof in the presence of acid, a reagent containing sulfur atom or an aluminum reagent if necessary, under the temperature ranging from room temperature to 300° C. The examples of acid include hydrogen chloride, p-toluenesulfonic acid and the like, and the reagent containing sulfur atom may include sulfur, hydrogen sulfide, carbon disulfide, phosphorus pentasulfide and the like.

The examples of the hydrocarbon solvent may include aromatic hydrocarbon such as benzene, toluene and the like, or aliphatic hydrocarbon such as pentane, hexane and the like. The alcohol solvent includes methanol, ethanol, propanol, 2-propanol, 2-methyl-2-propanol ethylene glycol, diethylene glycol and the like. The examples of ether solvent may include diethyl ether, dimethoxyethane, tetrahydrofuran, 1,4-dioxane and the like.

Examples of the aluminum reagent to be used in the reaction may include trimethylaluminum, triethylaluminum, dimethylaluminum chloride, diethylaluminum chloride, ethylaluminum dichloride and the like.

Process 2:

The compound (I) can be obtained by the reaction of the compound (IV) with the compound (V) in accordance with the following reaction scheme.

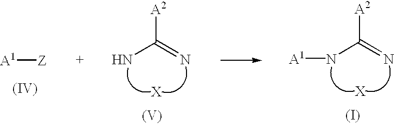

wherein, “Z” is leaving group which accelerates the reaction with nitrogen atoms of cyclic amidine compound, such as halogen atom, p-toluenesulfonyloxy, methanesulfonyloxy, trifluoromethane-sulfonyloxy, acyloxy, substituted acyloxy groups and so on.

The compounds (IV) and (V) to be used in this reaction can be commercially available or can be easily prepared from known compounds by using common methods.

The reaction of the compound (IV) with the compound (V) to obtain the compound (I) can be usually carried out in an appropriate solvent such as alcohol solvent, ketone solvent, nitrile solvent, ester solvent, amide solvent, hydrocarbon solvent and ether solvent or the mixture thereof in the presence of an organic base or an inorganic base if necessary, under the temperature ranging from −20° C. to the refluxing temperature of the solvent to be used.

The examples of alcohol solvent include methanol, ethanol, propanol, 2-propanol, 2-methyl-2-propanol and the like. The ketone solvent may include acetone, methyl ethyl ketone and the like. The nitrile solvent may include acetonitrile, propionitrile and so on, and the ester solvent may be ethyl acetate. The examples of amide solvent include N,N-dimethylformamide, N,N-dimethylacetamide, N-methylpyrrolidone, hexamethylphosphoramide and the like. The hydrocarbon solvent may include aromatic hydrocarbon such as benzene, toluene and the like, or aliphatic hydrocarbon such as pentane, hexane and the like. The examples of ether solvent may include diethyl ether, dimethoxyethane, tetrahydrofuran, 1,4-dioxane and the like.

Examples of the organic base to be used in the reaction may include triethylamine, collidine, lutidine, potassium tert-butoxide, sodium amide, lithium diisopropylamide, potassium bis(trimethylsilyl)amide and the like, and the inorganic base may include potassium carbonate, sodium carbonate, sodium hydrogencarbonate, sodium hydroxide, potassium hydroxide, sodium hydride, lithium hydride and the like.

Process 3:

The compound (I) can be obtained from the compound (VI) by the dehydrating cyclization of the compound (VI) in accordance with the following reaction scheme.

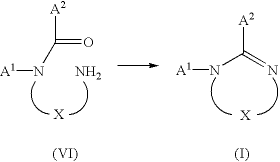

The compound (VI) to be used in this reaction can be prepared in accordance with the known method in this field.

This reaction can generally be carried out without solvent or in an appropriate solvent such as hydrocarbon solvent, halogenated hydrocarbon solvent and ether solvent, or in the mixture solvent thereof, in the presence of a dehydrating reagent if necessary, at the temperature ranging from −50° C. to 200° C., preferably from room temperature to 120° C.

The examples of hydrocarbon solvent may include aromatic hydrocarbon such as benzene, toluene and the like, or aliphatic hydrocarbon such as pentane, hexane and the like. The examples of halogenated hydrocarbon solvent may include dichloromethane, chloroform, 1,2-dichloroethane and the like. The ether solvent may include diethyl ether, dimethoxyethane, tetrahydrofuran, 1,4-dioxane and the like. The examples of the dehydrating reagent include thionyl chloride, sulfuryl chloride, phosphorus oxychloride, phosphorus trichloride, phosphorus pentachloride, p-toluenesulfonyl chloride, methanesulfonyl chloride, phosgene, diethyl azodicarboxylate, dicyclohexylcarbodiimide and the like.

The compound of formula (I) of the present invention thus obtained can be converted to a pharmaceutically acceptable salt with various kinds of the organic or inorganic acids mentioned above, if necessary. Furthermore, the compound (I) of the present invention can also be purified by the conventional manner, such as recrystallization, column chromatography and the like.

When the compounds of the formula (I) of the present invention exist in the isomer forms, each isomer per se is separated from each other by the conventional manner. Therefore, it is understood that each isomers per se, as well as the isomeric mixture, shall be included in the compounds of the present invention.

The compounds of formula (I) of the present invention bind selectively to nicotinic acetylcholine receptors in central nervous systems, and activate said receptors as agonists or modulators. Therefore, these compounds are useful as medicaments for preventing or treating various diseases, such as dementia, senile dementia, presenile dementia, Alzheimer's disease, Parkinson's disease, cerebrovascular dementia, AIDS-related dementia, dementia in Down's syndrome, Tourette's syndrome, neurosis during chronic cerebral infarction stage, cerebral dysfunction caused by cerebral injury, anxiety, schizophrenia, depression, Huntington's disease, pain and so on.

The compounds of formula (I) or a pharmaceutically acceptable salt thereof according to the present invention may be administered in the form of oral or parenteral formulations. The formulations for oral administration may include for example, tablets, capsules, granules, fine powders, syrups or the like; the formulations for parenteral administration may include, for example, injectable solutions or suspensions with distilled water for injection or other pharmaceutically acceptable solution, patches for transdermal application, sprays for nasally administration, depositories or the like.

These formulations may be formed by mixing with pharmaceutically acceptable carrier, excipient, sweeter, stabilizer and so on by the conventional procedures known per se to those skilled in the art in the field of pharmaceutical formulations.

Examples of pharmaceutically acceptable carrier or excipient include polyvinyl pyrrolidone, gum arabic, gelatin, sorbit, cyclodextrin, magnesium stearate, talc, polyethylene glycol, polyvinyl alcohol, silica, lactose, crystalline cellulose, sugar, starch, calcium phosphate, vegetable oil, carboxymethyl cellulose, hydroxypropyl cellulose, sodium lauryl sulfate, water, ethanol, glycerol, mannitol, syrup and the like.

The solutions for injection may be isotonic solution containing glucose and the like, and these solutions can be further contain an appropriate solubilizer such as polyethylene glycol or the like, buffer, stabilizer, preservative, antioxidant and so on.

These formulations can be administered to the human being and other mammalian animals, and the preferable administration route may Include oral route, transdermic route, nasal route, rectal route, topical route or the like.

The administration dose may vary in a wide range with ages, weights, condition of patients, routes of administration or the like, and a usual recommended daily dose to adult patients for oral administration is within the range of approximately 0.001-1,000 mg/kg per body weight, preferably 0.01-100 mg/kg per body weight, and more preferably 0.1-10 mg/kg per body weight.

In the case of parenteral administration such as intravenous injections, a usual recommended daily dose is within the range of approximately 0.00001-10 mg/kg per body weight, preferably 0.0001-1 mg/kg per body weight, and more preferably 0.001-0.1 mg/kg per body weight, once or in three times per day.

The methods for evaluating the binding capabilities of the compounds at nicotinic acetylcholine receptors are different by subtypes of receptors. The binding capabilities of the compounds at α4β2 nicotinic acetylcholine receptors are examined using rat brain membrane obtained from whole homogenized brain, and determining the inhibiting rate of the compounds against [³H]-cytisine binding to said brain membrane. Furthermore, the binding capabilities of the compounds at α1β1γδ nicotinic acetylcholine receptors are examined using homogenized rat muscle, and determining the inhibiting rate of the compounds against [³H]-α-bungarotoxin binding to said muscle homogenate.

The agonist effect in human α4β2 subtype of nicotinic acetylcholine receptors are examined by using human nicotinic acetylcholine receptors prepared in oocytes of Xenopus laevis, which is injected with cRNA from the corresponding cloning cDNA of human α4 and β2 subunits of nicotinic acetylcholine receptors, and to measure the expression of the electric response by adding the test compounds to perfusion solution by means of membrane potential holding method.

### EXAMPLES

The present invention is illustrated in more detail by way of the following examples.

### Example 1

### Synthesis by the Process 1

### 2-(6-chloro-3-pyridyl)methyl-1,4,5,6-tetrahydropyrimidine [Compound 8]

To a stirred solution of 20 ml of toluene were added 3.75 ml of 1M trimethylaluminum/hexane solution and 315 μl (3.77 mmol) of trimethylenediamine under argon atmosphere at room temperature, and to this mixture was further added 500 mg (2.5 mmol) of ethyl (6-chloro-3-pyridyl)acetate in toluene solution. The mixture was stirred for 22 hours at 100° C. under refluxing. After cooling the reaction mixture to room temperature, 5 ml of chloroform, 5 ml of methanol and 1 ml of water were added. Then precipitated gel was removed off by filtration and washed with a mixture of chloroform and methanol (9:1), and the filtrate was concentrated under reduced pressure. The resulting residue was purified by aminopropyl-coated silica gel (Chromatorex NH-type; Fuji Silysia Chemical Ltd.) column chromatography (eluent; dichloromethane:ethyl acetate=30:1, then dichloromethane:methanol=50:1) to give 442 mg (yield; 84.4%) of 2-(6-chloro-3-pyridyl)methyl-1,4,5,6-tetrahydropyrimidine as crystalline. This product was dissolved in methanol and to this solution was added 245 mg (2.11 mmol) of fumaric acid, and the mixture was concentrated under reduced pressure. The resulting oily residue was treated with acetonitrile to give crystalline. The crystalline was collected by filtration and dried in vacuum to give 643 mg of fumarate of the title Compound 8.

The following compounds were synthesized in accordance with the same procedures as described in Example 1.

Compound 1: 2-(6-chloro-3-pyridyl)-2-imidazoline;

Compound 2: 2-(6-chloro-3-pyridyl)-1,4,5,6-tetrahydropyrimidine;

Compound 3: 2-(6-chloro-3-pyridyl)-1-methyl-2-imidazoline;

Compound 4: 2-(6-chloro-3-pyridyl)-1-methyl-1,4,5,6-tetrahydropyrimidine;

Compound 6: 2-(6-chloro-3-pyridyl)imidazole;

Compound 7: 2-(6-chloro-3-pyridyl)methyl-2-imidazoline;

Compound 9: 2-(6-chloro-3-pyridyl)methyl-1-methyl-2-imidazoline;

Compound 10: 2-(6-chloro-3-pyridyl)methyl-1-methyl-1,4,5,6-tetrahydropyrimidine;

Compound 13: 2-(tetrahydrofuran-3-yl)-1,4,5,6-tetrahydropyrimidine;

Compound 14: 2-(tetrahydrofuran-3-yl)-2-imidazoline;

Compound 15: 2-(tetrahydrofuran-3-yl)methyl-1,4,5,6-tetrahydropyrimidine;

Compound 16: 2-(5-bromo-3-pyridyl)methyl-1,4,5,6-tetrahydropyrimidine;

Compound 17: 2-(5-bromo-3-pyridyl)methyl-2-imidazoline;

Compound 18: 2-(3-pyridyl)methyl-1,4,5,6-tetrahydropyrimidine;

Compound 19: 2-(3-pyridyl)methyl-2-imidazoline;

Compound 20: 2-(3-aminophenyl)-1,4,5,6-tetrahydropyrimidine;

Compound 21: 2-(3-quinolyl)methyl-1,4,5,6-tetrahydropyrimidine;

Compound 22: 2-(2-chloro-5-thiazolyl)-1,4,5,6-tetrahydropyrimidine;

Compound 23: 2-(3-quinolyl)methyl-2-imidazoline;

Compound 24: 2-(2-chloro-5-thiazolyl)-2-imidazoline;

Compound 25: 2-(3-quinolyl)-1,4,5,6-tetrahydropyrimidine;

Compound 26: 2-(3-furanyl)methyl-2-imidazoline;

Compound 28: 2-(3,5-dimethyl-4-isoxazolyl)methyl-1,4,5,6-tetrahydropyrimidine;

Compound 29: 2-(3,5-dimethyl-4-isoxazolyl)methyl-2-imidazoline;

Compound 30; 2-(3-thienyl)methyl-1,4,5,6-tetrahydropyrimidine;

Compound 31: 2-(3-thienyl)methyl-2-imidazoline;

Compound 33: 5-(3-pyridyl)-2-imidazoline;

Compound 36: 2-(5,6-dichloro-3-pyridyl)methyl-1,4,5,6-tetrahydropyrimidine;

Compound 37: 2-(6-chloro-3-pyridyl)methyl-5-phenyl-1,4,5,6-tetrahydropyrimidine;

Compound 38: 2-(4-pyridyl)methyl-1,4,5,6-tetrahydropyrimidine;

Compound 39: 2-(2-chloro-3-pyridyl)methyl-1,4,5,6-tetrahydropyrimidine;

Compound 40: 2-(2,6-dichloro-3-pyridyl)methyl-1,4,5,6-tetrahydropyrimidine;

Compound 41: 2-[2-(6-chloro-3-pyridyl)ethyl]-1,4,5,6-tetrahydropyrimidine;

Compound 42: 2-[2-(6-chloro-3-pyridyl)ethyl]-2-imidazoline;

Compound 43: 2-(6-methyl-3-pyridyl)methyl-1,4,5,6-tetrahydropyrimidine;

Compound 45: 2-(6-methyl-3-pyridyl)methyl-2-imidazoline;

Compound 46: 2-(6-ethoxy-3-pyridyl)methyl-1,4,5,6-tetrahydropyrimidine;

Compound 47: 2-(6-ethoxy-3-pyridyl)methyl-2-imidazoline;

Compound 48: 2-(6-fluoro-3-pyridyl)methyl-1,4,5,6-tetrahydropyrimidine;

Compound 49: 2-(5,6-dichloro-3-pyridyl)methyl-2-imidazoline;

Compound 50: 2-(6-chloro-3-pyridyl)methyl-5,5-dimethyl-1,4,5,6-tetrahydropyrimidine;

Compound 51: 2-(2-pyridyl)methyl-1,4,5,6-tetrahydropyrimidine;

Compound 53: 2-(5,6-dichloro-3-pyridyl)methyl-1-methyl-2-imidazoline;

Compound 54: 2-(6-chloro-3-pyridyl)methyl-4-methyl-1,4,5,6-tetrahydropyrimidine;

Compound 59: 3-(6-chloro-3-pyridyl)methyl-1,4,5,6-tetrahydro-1,2,4-triazine;

Compound 60: 2-[1-(6-chloro-3-pyridyl)ethyl]-1,4,5,6-tetrahydropyrimidine;

Compound 61: 1-(2-chloro-5-thiazolyl)methyl-1,4,5,6-tetrahydropyrimidine;

Compound 62: 1-[(2-(6-chloro-3-pyridyl)ethyl]-2-methyl-2-imidazoline;

Compound 63: 1-[2-(6-chloro-3-pyridyl)ethyl]-4,4-dimethyl-2-imidazoline;

Compound 64: 2-(2-chloro-5-thiazolyl)methyl-1,4,5,6-tetrahydropyrimidine;

Compound 65: 2-(2-chloro-5-thiazolyl)methyl-2-imidazoline;

Compound 66: 2-(5-pyrimidyl)methyl-1,4,5,6-tetrahydropyrimidine;

Compound 67: 2-(5-pyrimidyl)methyl-2-imidazoline;

Compound 68: 2-(5-methyl-3-pyridyl)methyl-1,4,5,6-tetrahydropyrimidine.

### Example 2

### Synthesis by the Process 2

### 1-(6-chloro-3-pyridyl)methyl-1,4,5,6-tetrahydropyrimidine [Compound 27]

To an ice-cooled solution of 384 mg (4.6 mmol) of 1,4,5,6-tetrahydropyrimidine in 5 ml of acetonitrile was added 619 mg (3 mmol) of 5-bromomethyl-2-chloropyridine, and the mixture was stirred for 15 minutes. After removal of solvent under reduced pressure, 6 ml of the solution of 0.5N potassium hydroxide in ethanol was added to the residue. The insoluble matter was removed off by filtration, and the filtrate was concentrated under reduced pressure. The resulting residue was dissolved in toluene, and the solvent was removed again under reduced pressure. The resulting residue was purified by aminopropyl-coated silica gel (Chromatorex NH-type; Fuji Silysia Chemical Ltd.) column chromatography (eluent; dichloromethane:methanol=40:1) to give 221 mg (yield; 35.2%) of 1-(6-chloro-3-pyridyl)methyl-1,4,5,6-tetrahydropyrimidine as colorless oil. This product was dissolved in methanol and to this solution was added 122 mg (1.05 mmol) of fumaric acid, and the mixture was concentrated under reduced pressure. The resulting residue was treated with acetonitrile to give crystalline. The crystalline was collected by filtration and dried in vacuum to give 308 mg of fumarate of the title Compound 27.

The following compounds were synthesized in accordance with the same procedures as described in Example 2.

Compound 5: 1-(6-chloro-3-pyridyl)methylimidazole;

Compound 10: 2-(6-chloro-3-pyridyl)methyl-1-methyl-1,4,5,6-tetrahydropyrimidine;

Compound 11: 1-(6-chloro-3-pyridyl)methyl-2-methyl-2-imidazoline;

Compound 34: 1,2-bis[(6-chloro-3-pyridyl)methyl]-1,4,5,6-tetrahydropyrimidine;

Compound 35: 1-(6-chloro-3-pyridyl)methyl-2-(3-pyridyl)-2-imidazoline;

Compound 44: 1,2-bis[(6-chloro-3-pyridyl)methyl]-2-imidazoline;

Compound 52: 1-(5,6-dichloro-3-pyridyl)methyl-1,4,5,6-tetrahydropyrimidine;

Compound 55: 1-[2-(6-chloro-3-pyridyl)ethyl]-1,4,5,6-tetrahydropyrimidine;

Compound 56: 1-(3-pyridazinyl)methyl-1,4,5,6-tetrahydropyrimidine;

Compound 57: 1-(6-methyl-3-pyridyl)methyl-1,4,5,6-tetrahydropyrimidine;

Compound 58: 1-(3-pyridyl)methyl-1,4,5,6-tetrahydropyrimidine;

Compound 61: 1-(2-chloro-5-thiazolyl)methyl-1,4,5,6-tetrahydropyrimidine;

Compound 62: 1-[2-(6-chloro-3-pyridyl)ethyl]-2-methyl-2-imidazoline;

Compound 63: 1-[2-(6-chloro-3-pyridyl)ethyl]-4,4-dimethyl-2-imidazoline.

### Example 3

### Synthesis by the Process 3

### 2-Methyl-5-(3-pyridyl)-2-imidazoline [Compound 32]

269 mg (1 mmol) of oxalate of N-[2-amino-1-(3-pyridyl)ethyl]acetamide was dissolved in 5 ml of phosphorus oxychloride, and this mixture was heated for 1.5 hours at 100° C. under stirring. After cooling the reaction mixture to room temperature, phosphorus oxychloride was removed off under reduced pressure. The resulting residue was treated with ice, and 1N sodium hydroxide aqueous solution was added to adjust the pH of the solution to 7, then, the mixture was concentrated under reduced pressure. The resulting residue was treated with ethanol and the insoluble matter was removed off by filtration, and the filtrate was concentrated under reduced pressure. The resulting residue was purified by aminopropyl-coated silica gel (Chromatorex NH-type; Fuji Silysia Chemical Ltd.) column chromatography (eluent; chloroform) to give 22 mg (yield; 13.6%) of 2-methyl-5-(3-pyridyl)-2-imidazoline as brownish oil. This product was dissolved in methanol and to this solution was added 15 mg (0.13 mmol) of fumaric acid, and the mixture was concentrated under reduced pressure. The resulting oily residue was treated with a mixture of t-butanol and acetone to give crystalline. The crystalline was collected by filtration and dried in vacuum to give 17 mg of fumarate of the title Compound 32.

The physicochemical data of the Compounds 1 to 68 obtained by the above-mentioned examples are summarized in the following Table 1 to Table 14.

TABLE 1

| | | | Properties | Mass Spectrum | |
|---|---|---|---|---|---|
| | | | m.p. (° C.) | found | |
| No. | Chemical Structure | Salt | crystallized solvent | molecular formula | ¹H-NMR (DMSO-d₆) |
| 1 | 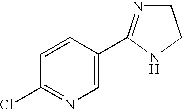 | fumarate | colorless cryst. 170-175° C. acetonitrile | m/z 182 = (M + H)⁺C₈H₈ClN₃ | 8.87(d, J=2.4Hz, 1H), 8.29(dd, J=2.4, 8.4Hz, 1H), 7.70(d, J=8.4Hz, 1H), 6.56(s, 2H), 3.78(s, 4H) |
| 2 | 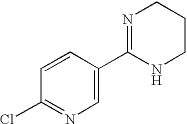 | fumarate | colorless cryst. 162-168° C. methanol/ acetonitrile | m/z 196 = (M + H)⁺C₉H₁₀ClN₃ | 8.79(d, J=2.5Hz, 1H), 8.24(dd, J=2.5, 8.3Hz, 1H), 7.74(d, J=8.3Hz, 1H), 6.40(s, 2H), 3.49(t, J=5.7Hz, 4H), 1.94(m, 2H) |
| 3 | 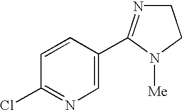 | fumarate | milky white cryst. 117-120° C. ether | m/z 196 = (M + H)⁺C₉H₁₀ClN₃ | 8.65(d, J=2.4Hz, 1H), 8.09(dd, J=2.4, 8.2Hz, 1H), 7.71(d, J=8.2Hz, 1H), 6.53(s, 2H), 3.84(m, 2H), 3.70(m, 2H), 2.89(s, 3H) |
| 4 | 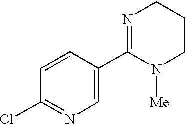 | oxalate | colorless oil | m/z 210 = (M + H)⁺C₁₀H₁₂ClN₃ | 10.26(br, 1H) 8.66(d, J=1.8Hz, 1H), 8.13(dd, J=1.8, 8.3Hz, 1H), 7.80(d, J=8.3Hz, 1H), 3.57(t, J=5.6Hz, 2H), 3.43(t, J=5.3Hz, 2H), 2.98(s, 3H), 2.08(m, 2H) |
| 5 | 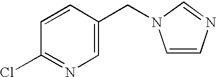 | fumarate | colorless cryst. 123-124° C. acetonitrile | m/z 194 = (M + H)⁺C₉H₈ClN₃ | 8.39(d, J=2.4Hz, 1H) 7.81(d, J=4.6Hz, 1H), 7.73 (dd, J=2.4, 8.2Hz, 1H), 7.52(d, J=8.2Hz, 1H), 7.24 (s, 1H), 6.94(br, 1H), 6.63(s, 2H), 5.26(s, 2H) |

TABLE 2

| | | | Properties | Mass Spectrum | |
|---|---|---|---|---|---|
| | | | m.p. (° C.) | found | |
| No. | Chemical Structure | Salt | crystallized solvent | molecular formula | ¹H-NMR(DMSO-d₆) |
| 6 | 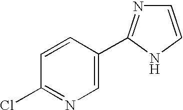 | fumarate | colorless cryst. 173-186° C. acetonitrile | m/z 180 = (M + H)⁺C₈H₆ClN₃ | 13.0(br, 3H), 8.94(d, J=2.5Hz, 1H), 8.30(dd, J=2.5, 8.3Hz, 1H), 7.60(d, J=8.3Hz, 1H), 7.23(s, 2H), 6.63(s, 2H) |
| 7 | 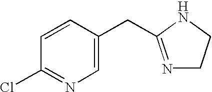 | fumarate | colorless cryst. 139-142° C. acetonitrile | m/z 196 = (M + H)⁺C₉H₁₀ClN₃ | 8.42(d, J=2.5Hz, 1H), 7.87(dd, J=2.5, 8.2Hz, 1H), 7.52(d, J=8.2Hz, 1H), 6.47(s, 2H), 3.93(s, 2H), 3.73(s, 4H) |
| 8 | 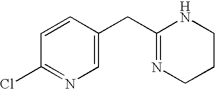 | fumarate | colorless cryst. 167-172° C. acetonitrile | m/z 210 = (M + H)⁺C₁₀H₁₂ClN₃ | 8.46(d, J=2.5Hz, 1H), 7.92(dd, J=2.5, 8.3Hz, 1H), 7.52(d, J=8.3Hz, 1H), 6.45(s, 2H), 3.87(s, 2H), 3.32(t, J=5.7Hz, 4H), 1.81(m, 2H) |
| 9 | 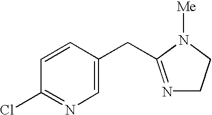 | fumarate | colorless cryst. 123-126° C. acetonitrile | m/z 210 = (M + H)⁺C₁₀H₁₂ClN₃ | 8.43(br, 1H), 7.86(dd, J=2.3, 8.2Hz, 1H), 7.54(d, J=8.2Hz, 1H), 6.48(s, 2H), 4.06(s, 2H), 3.76(m, 4H), 3.00(s,3H) |
| 10 | 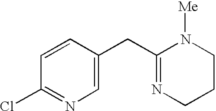 | oxalate | colorless cryst. 85-89° C. acetone | m/z 224 = (M + H)⁺C₁₁H₁₄ClN₃ | 8.42(d, J=2.4Hz, 1H), 7.84(dd, J=2.4, 8.2Hz, 1H), 7.55(d, J=8.2Hz, 1H), 4.07(s, 2H), 3.44(t, J=5.7Hz, 2H), 3.35(t, J=5.7Hz, 2H), 3.06(s, 3H), 1.95(m, 2H) |

TABLE 3

| | | | Properties | Mass Spectrum | |
|---|---|---|---|---|---|
| | | | m.p. (° C.) | found | |
| No. | Chemical Structure | Salt | crystallized solvent | molecular formula | ¹H-NMR(DMSO-d₆) |
| 11 | 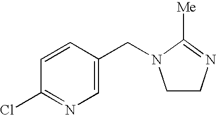 | fumarate | colorless cryst. 165-171° C. acetonitrile | m/z 210 = (M + H)⁺C₁₀H₁₂ClN₃ | 8.45(d, J=2.5Hz, 1H), 7.89(dd, J=2.5, 8.2Hz, 1H), 7.57(d, J=8.2Hz, 1H), 6.46(s, 2H), 4.63(s, 2H), 3.73(m, 2H), 3.63(m, 2H), 2.32(s, 3H) |
| 12 | 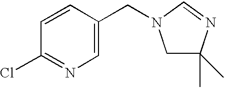 | fumarate | colorless cryst. 166-168° C. acetonitrile | m/z 224 = (M + H)⁺C₁₁H₁₄ClN₃ | 8.41(d, J=2.5Hz, 1H), 7.95(s, 1H), 7.86(dd, J=2.5, 8.2Hz, 1H), 7.56(d, J=8.2Hz, 1H), 6.49(s, 2H), 4.57(s, 2H), 3.17(s, 2H), 1.24(s, 6H) |
| 13 | 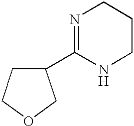 | fumarate | pale yellow cryst. 54-57° C. acetone | m/z 155 = (M + H)⁺C₈H₁₄N₂O | 9.9(br, 1H), 6.43(s, 2H), 3.88(m, 2H), 3.72(m, 2H), 3.31(t, J=5.7Hz, 4H), 3.29(m, 1H), 2.21(m, 1H), 2.04(m, 1H), 1.84(quintet, J=5.7Hz, 2H) |
| 14 | 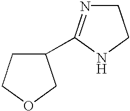 | fumarate | colorless cryst. 103-105° C. acetone | m/z 141 = (M + H)⁺C₇H₁₂N₂O | 6.43(s, 2H), 3.86(m, 2H), 3.73(s, 4H), 3.72(m, 2H), 3.35(m, 1H), 2.19(m, 1H), 2.06(m, 1H) |
| 15 | 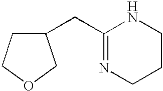 | oxalate | colorless cryst. 187-190° C. acetone | m/z 169 = (M + H)⁺C₉H₁₈N₂O | 9.71(br, 2H), 3.74(m, 2H), 3.64(m, 1H), 3.32(m, 4H), 2.44(m, 4H), 1.99(m, 1H), 1.84(m, 2H), 1.54(m, 1H) |

TABLE 4

| | | | Properties | Mass Spectrum | |
|---|---|---|---|---|---|
| | | | m.p. (° C.) | found | |
| No. | Chemical Structure | Salt | crystallized solvent | molecular formula | ¹H-NMR (DMSO-d₆) |
| 16 | 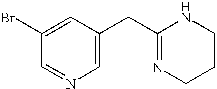 | fumarate | colorless cryst. 155-159° C. acetone | m/z 254 = (M + H)⁺C₁₀H₁₂BrN₃ | 8.66(d, J=1.6Hz, 1H), 8.62(d, J=1.6Hz, 1H), 8.16 (s, 1H), 6.39(s, 2H), 3.87(s, 2H), 3.33(m, 4H), 1.81 (m, 2H) |
| 17 | 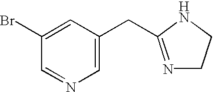 | fumarate | colorless cryst. 150-154° C. acetone | m/z 242 = (M + H)⁺C₉H₁₀BrN₃ | 8.63(s, 1H), 8.53(s, 1H), 8.05(s, 1H), 6.44(s, 2H), 3.78(s, 2H), 3.65(s, 4H) |
| 18 | 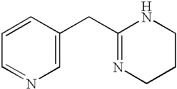 | fumarate | colorless cryst. 120-124° C. ethanol/ acetone | m/z 176 = (M + H)⁺C₁₀H₁₃N₃ | 10.77(2H, br), 8.62(1H, s), 8.51(d, J=4.8Hz, 1H), 7.85(d, J=7.6Hz, 1H), 7.39(dd, J=4.8, 7.6Hz, 1H), 6.42(s, 2H), 3.86(s, 2H), 3.33(m, 4H), 1.81(m, 2H) |
| 19 | 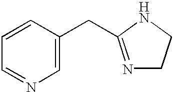 | fumarate | colorless cryst. 134-135° C. acetone | m/z 162 = (M + H)⁺C₉H₁₁N₃ | 8.57(d, J=2.0Hz, 1H), 8.51(dd, J=2.0, 4.7Hz, 1H), 7.78(d, J=7.8Hz, 1H), 7.39(dd, J=4.7, 7.8Hz, 1H), 6.46(s, 2H), 3.85(s, 2H), 3.72(s, 4H) |
| 20 | 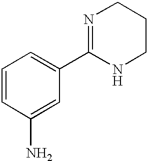 | fumarate | colorless cryst. 192-195° C. acetone | m/z 176 = (M + H)⁺C₁₀H₁₃N₃ | 7.21(m, 1H), 6.85(s, 1H), 6.81(m, 2H), 6.37(s, 2H), 5.54(br, 2H), 3.45(m, 4H), 1.95(m, 2H) |

TABLE 5

| | | | Properties | Mass Spectrum | |
|---|---|---|---|---|---|
| | | | m.p. (° C.) | found | |
| No. | Chemical Structure | Salt | crystallized solvent | molecular formula | ¹H-NMR(DMSO-d₆) |
| 21 | 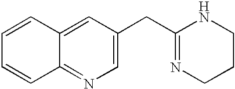 | fumarate | colorless cryst. 168-171° C. acetone | m/z 226 = (M + H)⁺C₁₄H₁₅N₃ | 8.94(s, 1H), 8.38(s, 1H), 8.03(d, J=8.4Hz, 1H), 7.94(d, J=8.1Hz, 1H), 7.77(m, 1H), 7.64(m, 1H), 6.42(s, 2H), 4.05(s, 2H), 3.34(m, 4H), 1.83(m, 2H) |
| 22 | 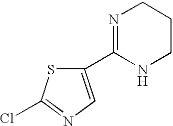 | fumarate | colorless cryst. 159-160° C. acetone | m/z 202 = (M + H)⁺C₇H₈ClN₃S | 8.03(s, 1H), 6.56(s, 2H), 3.34(m, 4H), 1.76(m, 2H) |
| 23 | 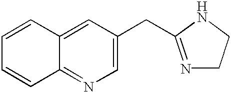 | fumarate | colorless cryst. 175-177° C. acetone | m/z 212 = (M + H)⁺C₁₃H₁₃N₃ | 8.88(s, 1H), 8.31(s, 1H), 8.03(d, J=8.4Hz, 1H), 7.96(d, J=8.1Hz, 1H), 7.78(m, 1H), 7.64(m, 1H), 6.47(s, 2H), 4.06(s, 2H), 3.75(s, 4H) |
| 24 | 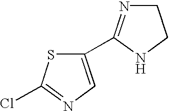 | fumarate | pale yellow cryst. 157-158° C. acetone | m/z 188 = (M + H)⁺C₆H₆ClN₃ | 8.02(s, 1H), 6.62(s, 2H), 3.62(s, 4H) |
| 25 | 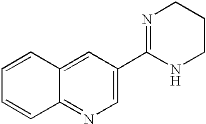 | fumarate | colorless cryst. 188-193° C. acetone | m/z 212 = (M + H)⁺C₁₃H₁₃N₃ | 9.16(d, J=2.2Hz, 1H), 8.82(d, J=2.2Hz, 1H), 8.13(m, 2H), 7.95(m, 1H), 7.76(m, 1H), 6.38(s, 2H), 3.55(m, 4H), 2.00(m, 2H) |

TABLE 6

| | | | Properties | Mass Spectrum | |
|---|---|---|---|---|---|
| | | | m.p. (° C.) | found | |
| No. | Chemical Structure | Salt | crystallized solvent | molecular formula | ¹H-NMR(DMSO-d₆) |
| 26 | 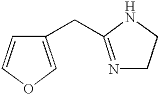 | fumarate | colorless cryst. 200-205° C. acetone | m/z 151 = (M + H)⁺C₈H₁₀N₂O | 7.66(s, 1H), 7.64(s, 1H), 6.50(s, 1H), 6.41(s, 2H), 3.74(s, 4H), 3.69(s, 2H), |
| 27 | 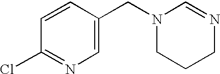 | fumarate | colorless cryst. 126-129° C. acetonitrile | m/z 210 = (M + H)⁺C₁₀H₁₂ClN₃ | 8.47(m, 2H), 7.92(dd, J=2.5, 8.2Hz, 1H), 7.59(d, J=8.2Hz, 1H), 6.44(s, 2H), 4.69(s, 2H), 3.25(m, 4H), 1.88(m, 2H) |
| 28 | 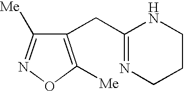 | fumarate | colorless cryst. 188-190° C. acetone | m/z 194 = (M + H)⁺C₁₀H₁₅N₃O | 10.37(br, 2H), 6.39(s, 2H), 3.68(s, 2H), 3.32(m, 4H), 2.34(s, 3H), 2.14(s, 3H), 1.83(m, 2H) |
| 29 | 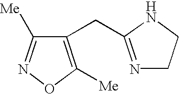 | fumarate | colorless cryst. 208-215° C. ethanol | m/z 180 = (M + H)⁺C₉H₁₃N₃O | 6.43(s, 2H), 3.72(s, 4H), 3.64(s, 2H), 2.34(s, 3H), 2.14(s, 3H) |
| 30 | 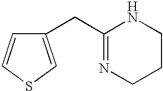 | fumarate | colorless cryst. 85-90° C. acetone | m/z 181 = (M + H)⁺C₉H₁₂N₂S | 7.55(d, J=4.8Hz, 1H), 7.46(s, 1H), 7.13(d, J=4.8Hz, 1H), 6.40(s, 2H), 3.78(s, 2H), 3.33(m, 4H), 1.83(m, 2H) |

TABLE 7

| | | | Properties | Mass Spectrum | |
|---|---|---|---|---|---|
| | | | m.p. (° C.) | found | |
| No. | Chemical Structure | Salt | crystallized solvent | molecular formula | ¹H-NMR(DMSO-d₆) |
| 31 | 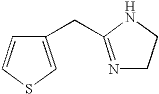 | fumarate | colorless cryst. 150-153° C. acetone | m/z 167 = (M + H)⁺C₈H₁₀N₂S | 7.55(d, J=4.8Hz, 1H), 7.43(s, 1H), 7.11(d, J=4.8Hz, 1H), 6.43(s, 2H), 3.83(s, 2H), 3.75(s, 4H) |
| 32 | 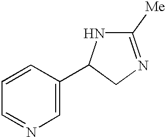 | fumarate | pale brown cryst. 130-132° C. t-butanol/ acetone | m/z 162 = (M + H)⁺C₉H₁₁N₃ | 8.60(s, 1H), 8.57(m, 1H), 7.81(d, J=6.8Hz, 1H), 7.45(m, 1H), 6.48(s, 2H), 5.33(m, 1H), 4.23(m, 1H), 3.64(m, 1H), 2.24(s, 3H) |
| 33 | 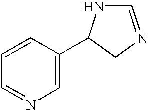 | fumarate | colorless cryst. 148-149° C. ethanol/ acetone | m/z 148 = (M + H)⁺C₈H₉N₃ | 8.56(m, 2H), 8.14(s, 1H), 7.75(d, J=7.0Hz, 1H), 7.43(m, 1H), 6.54(s, 2H), 5.24(m, 1H), 4.15(m, 1H), 3.55(m, 1H) |
| 34 | 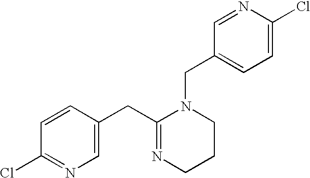 | fumarate | pale brown cryst. 135-139° C. acetonitrile | m/z 335 = (M + H)⁺C₁₆H₁₆Cl₂N₄ | 8.40(d, J=2.3Hz, 1H), 8.20(s, 1H), 7.84(dd, J=2.3, 8.3Hz, 1H), 7.64(d, J=8.2Hz, 1H), 7.47(m, 2H), 6.47(s, 2H), 4.74(s, 2H), 4.23(s, 2H), 3.42(t, J=5.4Hz, 2H), 3.34(t, J=5.3Hz, 2H), 1.96(m, 2H) |
| 35 | 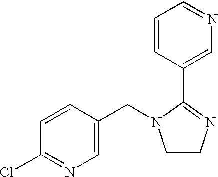 | fumarate | pale brown cryst. 164-166° C. acetone | m/z 273 = (M + H)⁺C₁₄H₁₃ClN₄ | 8.76(d, J=1.8Hz, 1H), 8.71(dd, |
| # J=1.5, 4.8Hz, 1H), 8.34(d, J=2.4Hz, 1H), 7.97(ddd, J=1.5, 1.8, 7.8Hz, 1H), 7.81(dd, J=2.4, 8.2Hz, 1H), 7.53(dd, J=4.8, 7.8Hz, 1H), 7.52(d, J=8.2Hz, 1H), 6.58(s, 2H), 4.32(s, 2H), 3.83(t, J=10.0Hz, 2H), 3.45(t, J=10.0Hz, 2H) | | | | | |

TABLE 8

| | | | Properties | Mass Spectrum | |
|---|---|---|---|---|---|
| | | | m.p. (° C.) | found | |
| No. | Chemical Structure | Salt | crystallized solvent | molecular formula | ¹H-NMR(DMSO-d₆) |
| 36 | 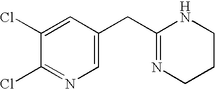 | fumarate | colorless cryst. 198-200° C. acetone | m/z 244 = (M + H)⁺C₁₀H₁₁Cl₂N₃ | 8.31(d, J=2.1Hz, 1H), 8.01(d, J=2.1Hz, 1H), 6.88(s, 2H), 3.85(s, 2H), 3.43(m, 4H), 1.99(m, 2H) in CD₃OD |
| 37 | 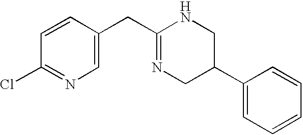 | fumarate | colorless cryst. 163-168° C. acetone | m/z 286 = (M + H)⁺C₁₆H₁₈ClN₃ | 8.49(d, J=2.4Hz, 1H), 7.94(dd, J=2.4, 8.2Hz, 1H), 7.55(d, J=8.2Hz, 1H), 7.30(m, 5H), 6.44(s, 2H), 3.94(s, 2H), 3.57(m, 2H), 3.45(m, 2H), 3.08(m, 1H) |
| 38 | 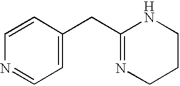 | fumarate | colorless cryst. 141-143° C. acetone | m/z 176 = (M + H)⁺C₁₀H₁₃N₃ | 8.55(d, J=5.8Hz, 2H), 7.40(d, J=5.8Hz, 2H), 6.48(s, 2H), 3.84(s, 2H), 3.34(t, J=5.7Hz, 4H), 1.83(m, 2H) |
| 39 | 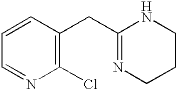 | fumarate | colorless cryst. 160-161° C. acetone | m/z 210 = (M + H)⁺C₁₀H₁₂ClN₃ | 8.38(dd, J=1.7, 4.8Hz, 1H), 7.89(dd, J=1.7, 7.6Hz, 1H), 7.46(dd, J=4.8, 7.6Hz, 1H), 6.35(s, 2H), 3.97(s, 2H), 3.35(t, J=5.7Hz, 4H), 1.87(m, 2H) |
| 40 | 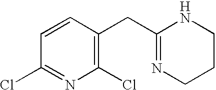 | fumarate | colorless cryst. 175-177° C. acetone | m/z 244 = (M + H)⁺C₁₀H₁₁Cl₂N₃ | 7.86(d, J=8.0Hz, 1H), 7.50(d, J=8.0Hz, 1H), 6.68(s, 2H), 3.97(s, 2H), 3.45(t, J=5.7Hz, 4H), 2.02(m, 2H) in CD₃OD |

TABLE 9

| | | | Properties | Mass Spectrum | |
|---|---|---|---|---|---|
| | | | m.p.(° C.) | found | |
| No. | Chemical Structure | Salt | crystallized solvent | molecular formula | ¹H-NMR(DMSO-d₆) |
| 41 | 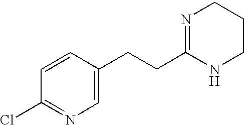 | fumarate | colorless cryst. 156-157° C. acetone | m/z 224 = (M + H)⁺C₁₁H₁₄ClN₃ | 8.28(s, 1H), 7.74(d, J=8.2Hz, 1H), 7.46(d, J=8.2Hz, 1H), 6.70(s, 2H), 3.41(t, J=5.5Hz, 4H), 3.02(t, J=7.6Hz, 2H), 2.73(t, J=7.6Hz, 2H), 1.95(m, 2H) in CD₃OD |
| 42 | 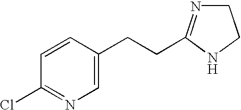 | fumarate | colorless cryst. 148-149° C. acetone | m/z 210 = (M + H)⁺C₁₀H₁₂ClN₃ | 8.27(s, 1H), 7.73(d, J=8.0Hz, 1H), 7.43(d, J=8.0Hz, 1H), 6.68(s, 2H), 3.90(s, 4H), 3.02(br, 2H), 2.86(br, 2H), 2.86(br, 2H) in CD₃OD |
| 43 | 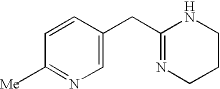 | fumarate | colorless cryst. 156-158° C. 2-propanol/ acetone | m/z 190 = (M + H)⁺C₁₁H₁₅N₃ | 8.46(s, 1H), 7.71(d, J=7.9Hz, 1H), 7.23(d, J=7.9Hz, 1H), 6.40(s, 2H), 3.77(s, 2H), 3.31(m, 4H), 2.44(s, 3H), 1.80(m, 2H) |
| 44 | 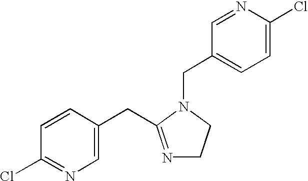 | fumarate | milky white cryst. 162-164° C. 2-propanol | m/z 321 = (M + H)⁺C₁₅H₁₄Cl₂N₄ | 8.38(d, J=2.0Hz, 1H), 8.31(d, J=2.4Hz, 1H), 7.82(dd, J=2.0, 8.2Hz, 1H), 7.75(dd, J=2.4, 8.2Hz, 1H), 7.51(d, J=8.2Hz, 1H), 7.49(d, J=8.2Hz, 1H), 6.52(s, 2H), 4.57(s, 2H), 4.00(s, 2H), 3.68(m, 2H), 3.47(m, 2H) |
| 45 | 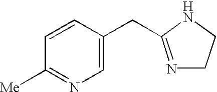 | fumarate | colorless cryst. 165-166° C. acetone | m/z 176 = (M + H)⁺C₁₀H₁₃N₃ | 8.42(d, J=2.2Hz, 1H), 7.66(dd, J=2.2, 8.0Hz, 1H), 7.23(d, J=8.0Hz, 1H), 6.44(s, 2H), 3.82(s, 2H), 3.72(s, 4H), 2.44(s, 3H) |

TABLE 10

| | | | | Mass Spectrum | |
|---|---|---|---|---|---|
| | | | m.p. (° C.) | found | |
| No. | Chemical Structure | Salt | crystallized solvent | molecular formula | ¹H-NMR(DMSO-d₆) |
| 46 | 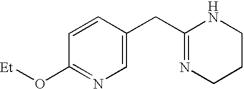 | fumarate | colorless cryst. 110-112° C. acetone | m/z 220 = (M + H)³⁰C₁₂H₁₇N₃O | 8.16(d, J=2.3Hz, 1H), 7.72(dd, J=2.3, 8.5Hz, 1H), 6.78(d, J=8.5Hz, 1H), 6.39(s, 2H), 4.28(q, J=7.0Hz, 2H), 3.72(s, 2H), 3.31(t, J=5.7Hz, 4H), 1.80(m, 2H), 1.30(t, J=7.0Hz,m 3H) |
| 47 | 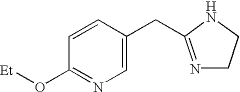 | fumarate | colorless cryst. 170-171° C. acetone | m/z 206 = (M + H)³⁰C₁₁H₁₅N₃O | 8.12(d, J=2.2Hz, 1H), 7.68(dd, J=2.2, 8.5Hz, 1H), 6.78(d, J=8.5Hz, 1H), 6.42(s, 2H), 4.27(q, J=7.0Hz, 2H), 3.76(s, 2H), 3.72(s, 4H), 130(t, J=7.0Hz, 3H) |
| 48 | 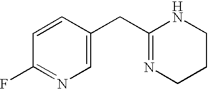 | fumarate | pale yellow cryst. 136-139° C. acetone | m/z 194 = (M + H)³⁰C₁₀H₁₂FN₃ | 8.27(s, 1H), 8.03(ddd, J=2.3, 8.2, 8.4Hz, 1H), 7.21(dd, J=8.4, 2,7Hz, 1H), 6.39(s, 2H), 3.84(s, 2H), 3.32(t, J=5.7, 4H), 1.81(m, 2H) |
| 49 | 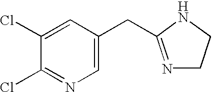 | fumarate | colorless cryst. 176-178° C. acetone | m/z 230 = (M + H)³⁰C₉H₉Cl₂N₃ | 8.37(s, 1H), 8.15(s, 1H), 6.46(s, 2H), 3.85(s, 2H), 21 3.66(s, 4H) |
| 50 | 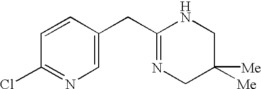 | fumarate | pale yellow cryst. 143-145° C. acetone | m/z 238 = (M + H)³⁰C₁₂H₁₆ClN₃ | 8.37(s, 1H), 7.82(dd, J=2.4, 8.2Hz, 1H), 7.50(d, J=8.2Hz, 1H), 6.68(s, 2H), 3.86(s, 2H), 3.13(s, 4H), 1.02(s, 6H) in CD₃OD |

TABLE 11

| | | | Properties | Mass Spectrum | |
|---|---|---|---|---|---|
| | | | m.p. (° C.) | found | |
| No. | Chemical Structure | Salt | crystallized solvent | molecular formula | ¹H-NMR(DMSO-d₆) |
| 51 | 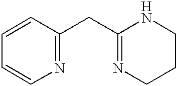 | fuma- rate | milky white cryst. 120-122° C. acetone | m/z 176 = (M + H)⁺C₁₀H₁₃N₃ | 8.56(d, J=4.7Hz, 1H), 7.84(t, J=7.0, 7.8Hz, 1H), 7.41(d, J=7.8Hz, 1H), 7.37(t, J=4.7, 7.0Hz, 1H), 6.70(s, 2H), 3.95(s, 2H), 3.48(t, J=5.7Hz, 4H), 2.01(q, J=5.7Hz, 2H), in CD₃OD |
| 52 | 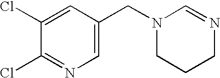 | fuma- rate | colorless cryst. 185-186° C. acetone | m/z 244 = (M + H)⁺C₁₀H₁₁Cl₂N₃ | 8.37(d, J=2.1Hz, 1H), 8.33(s, 1H), 8.10(d, J=2.1Hz, 1H), 6.68(s, 2H), 4.70(s, 2H), 3.31(m, 4H), 2.04(m, 2H) in CD₃OD |
| 53 | 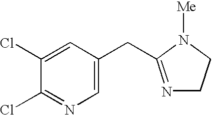 | fuma- rate | colorless cryst. 152° C. acetone | m/z 244 = (M + H)⁺C₁₀H₁₁Cl₂N₃ | 8.36(d, J=2.1Hz, 1H), 8.06(d, J=2.1Hz, 1H), 6.71 (s, 2H), 4.01(t, J=11.5Hz, 2H), 3.80(t, J=11.5Hz, 2H), 3.34(s, 2H), 3.20(s, 3H), in CD₃OD |
| 54 | 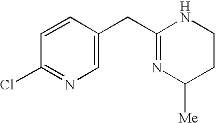 | fuma- rate | colorless cryst. 157° C. acetone | m/z 224 = (M + H)⁺C₁₁H₁₄ClN₃ | 8.37(d, J=2.5Hz, 1H), 7.81(m, 1H), 7.51(m, 1H), 6.70(s, 2H), 3.83(s, 2H), 3.69(m, 1H), 3.45(m, 2H), 2.11(m, 1H), 1.68(m, 1H), 1.34(m, 3H) in CD₃OD |
| 55 | 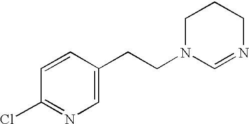 | fuma- rate | pale yellow cryst. 138-143° C. acetone | m/z 224 = (M + H)⁺C₁₁H₁₄ClN₃ | 8.34(s, 1H), 8.03(s, 1H), 7.81(d, J=8.1Hz, 1H), 7.50(d, J=8.1Hz, 1H), 6.37(s, 2H), 3.67(t, J=6.9Hz, 2H), 3.42(m, 2H), 3.22(m, 2H), 2.95(t, J=6.9Hz, 2H), 1.89(m, 2H) |

TABLE 12

| | | | Properties | Mass Spectrum | |
|---|---|---|---|---|---|
| | | | m.p. (° C.) | found | |
| No. | Chemical Structure | Salt | crystallized solvent | molecular formula | ¹H-NMR(DMSO-d₆) |
| 56 | 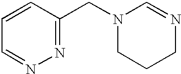 | fumarate (1.5 molecules) | colorless cryst. 124-125° C. acetone | m/z 177 = (M + H)⁺C₉H₁₂N₄ | 9.22(s, 1H), 8.37(s, 1H), 7.80(s, 1H), 7.79(s, 1H), 6.71(s, 3H), 5.01(s, 2H), 3.49(t, J=5.5Hz, 2H), 3.43(t, J=5.5Hz, 2H), 2.11(t, J=5.5Hz, 2H) in CD₃OD |
| 57 | 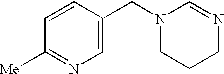 | fumarate (2 molecules) | colorless cryst. 156-157° C. acetone | m/z 190 = (M + H)⁺C₁₁H₁₅N₃ | 8.49(s, 2H), 7.72(d, J=7.8Hz, 1H), 7.32(d, J=7.8Hz, 1H), 6.53(s, 4H), 4.65(s, 2H), 3.25(m, 4H), 2.50(s, 3H), 1.87(m, 2H) |
| 58 | 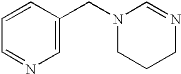 | fumarate (2 molecules) | colorless cryst. 141-142° C. acetone | m/z 176 = (M + H)⁺C₁₀H₁₃N₃ | 8.62(s, 1H), 8.58(d, J=4.8Hz, 1H), 8.49(s, 1H), 7.83(d, J=7.7Hz, 1H), 7.48(dd, J=4.8, 7.7Hz, 1H), 6.52(s, 4H), 4.59(s, 2H), 3.25(m, 4H), 1.87(m, 2H) |
| 59 | 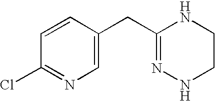 | hydro- chloride (2 molecules) | yellow cryst. 134-140° C. acetonitrile | m/z 211 = (M + H)⁺C₄H₁₁ClN₄ | 11.46(br, 1H), 10.21(br, 1H), 8.47(s, 1H), 7.93(d, J=8.2Hz, 1H), 7.57(d, J=8.2Hz, 1H), 5.94(br, 1H), 3.81(s, 2H), 3.38(m, 2H), 3.00(m, 2H) |
| 60 | 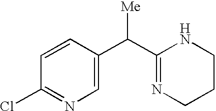 | fumarate | colorless cryst. 156-158° C. acetone | m/z 224 = (M + H)⁺C₁₁H₁₄ClN₃ | 8.37(d, J=2.5Hz, 1H), 7.81(dd, J=2.5, 8.3Hz, 1H), 7.50(d, J=8.3Hz, 1H), 6.68(s, 2H), 4.04(q, J=7.2Hz, 1H), 3.45(t, J=5.7Hz, 4H), 1.98(quintet, J=5.7Hz, 2H), 1.63(d, J=7.2Hz, 3H), in CD₃OD |

TABLE 13

| | | | Properties | | |
|---|---|---|---|---|---|
| | | | m.p. (° C.) | Mass Spectrum | |
| | | | crystallized | found | |
| No. | Chemical Structure | Salt | solvent | molecular formula | ¹ H-NMR(DMSO-d₆) |
| 61 | 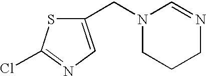 | fumarate | colorless cryst 133-134° C. acetone | m/z 216 = (M + H)⁺C₉H₁₀ClN₃S | 8.11(s, 1H), 7.66(s, 1H), 6.41(s, 2H), 4.56(s, 2H), 3.35(m, 4H), 1.77(m, 2H) |
| 62 | 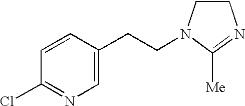 | fumarate | colorless cryst. 144-146° C. acetone | m/z 224 = (M + H)⁺C₁₁H₁₄ClN₃ | 8.38(d, J=2.1Hz, 1H), 7.85(dd, J=2.1, 8.2Hz, 1H), 7.50(d, J=8.2Hz, 1H), 6.38(s, 2h), 3.75(m, 4H), 3.59(t, J=7.2Hz, 2H), 2.91(t, J=7.2Hz, 2H), 2.09(s, 3H) |
| 63 | 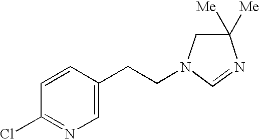 | hydro- chloride (2 molecules) | colorless cryst. 158-162° C. acetone | m/z 238 = (M + H)⁺C₁₂H₁₆ClN₃ | 10.34(1H, s), 8.36(d, J=2.4Hz, 1H), 8.28(1H, s), 7.81(dd, J=2.4, 8.2Hz, 1H), 7.52(d, J=8.2Hz, 1H), 3.74(t, J=6.8Hz, 4H), 3.62(s, 2H), 2.97(t, J=6.8Hz, 2H), 2.09(s, 3H), 1.31(s, 3H) |
| 64 | 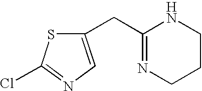 | hydro- chloride (2 molecules) | colorless cryst. 213-220° C. acetone | m/z 216 = (M + H)⁺C₈H₁₀ClN₃S | 10.06(s, 2H), 7.70(s, 1H), 4.07(s, 2H), 3.32(m, 4H), 1.82(m, 2H) |
| 65 | 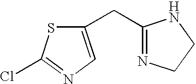 | fumarate | yellow cryst. 148-150° C. acetone | m/z 202 = (M + H)⁺C₇H₈ClN₃S | 7.58(s, 1H), 6.49(s, 2H), 4.03(s, 2h), 3.65(s, 4H) |

TABLE 14

| | | | Properties | Mass Spectrum | |
|---|---|---|---|---|---|
| | | | m.p. (° C.) | found | |
| No. | Chemical Structure | Salt | crystallized solvent | molecular formula | ¹H-NMR(DMSO-d₆) |
| 66 | 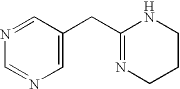 | fumarate | colorless cryst. 151-156° C. acetone | m/z 177 = (M + H)⁺C₉H₁₂N₄ | 9.13(s, 1H), 8.85(s, 2H), 6.43(s, 2H), 3.90(s, 2H), 3.33(m, 4H), 1.82(m, 2H) |
| 67 | 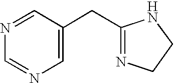 | fumarate | colorless cryst. 155-156° C. acetone | m/z 163 = (M + H)⁺C₈H₁₀N₄ | 9.12(s, 1H), 8.80(s, 2h), 6.46(s, 2h), 3.89(s, 2H), 3.71(s, 4) |
| 68 | 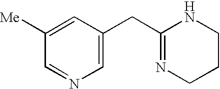 | fumarate | colorless cryst. 137-139° C. acetone | m/z 190 = (M + H)⁺C₁₁H₁₅N₃ | 10.42(s, 2H), 8.40(s, 1h), 8.35(s, 1H), 7.63(s, 1H), 6.47(s, 2H), 3.78(s, 1H), 3.33(m, 4H), 2.29(s, 3H), 1.81(m, 2H) |

The effect of the compounds (I) of the present invention was evaluated by the following biological experiments.

Biological Experiment 1:

Binding Assays at α4β2 Subtype of Nicotinic Acetylcholine Receptors

The affinity of the compounds of the present invention to α4β2 subtype of nicotinic acetylcholine receptors was performed by the following method, which was modified method described by Pabreza L. A., Dhawan S. & Kellar K. J., Mol. Pharm., 39, 9-12 (1990), and by Anderson D. J. & Arneric S. P., Eur. J. Pharm., 253, 261-267 (1994).

(1) Preparation of Rat Brain Membrane Containing α4β2 Subtype of Nicotinic Acetylcholine Receptors

Fischer-344 strain male rats (body weight: 200-240 g; 9 weeks old) obtained from Charles River Japan were used. Rats were housed in the breeding cage controlled of the room temperature at 23±1° C., and the humidity of 55±5% for 1 to 4 weeks. Rats (3 to 4 rats per a cage) were housed with lights on for 12 hours daily (from 7:00 to 19:00), and allowed free access to food and water.

Preparation of rat brain membrane containing α4β2 subtype of nicotinic acetylcholine receptors was performed as follow. That is, rat brains were isolated just after sacrificed by decapitation, washed with ice-cooled saline solution and then frozen at −80° C. with liquid nitrogen and stored till using. After thawing the frozen brain, the brain was homogenized in 10 volumes of ice-cooled buffer solution (50 mM of Tris-HCl, 120 mM of NaCl, 5 mM of KCl, 1 mM of MgCl₂, 2 mM of CaCl₂; pH 7.4; 4° C.) using homogenizer (HG30, Hitachi Kohki Ltd.) for 30 seconds, and the homogenate were centrifuged under 1,000×G for 10 minutes at 4° C. The resulting supernatant was separated and the pellet was homogenized again with half volume of aforementioned prior buffer solution and centrifuged under the same conditions. Combined supernatant was further centrifuged under 40,000×G for 20 minutes at 4° C. The pellet was suspended in buffer solution and used for binding assays at receptors.

(2) Experiments of α4β2 Subtype of Nicotinic Acetylcholine Receptors Binding

Suspensions of membrane pellets containing 400-600 μg of protein were added to test tubes containing test compounds and [³H]-cytisine (2 nM) in a final volume of 200 μl and incubated for 75 minutes in ice-cooled bath. The samples were isolated by vacuum filtration onto Whatman GF/B filters, which were prerinsed with 0.5% polyethylenimine just prior to sample filtration, using Brandel multi manifold cell harvester. The filters were rapidly washed with buffer solution (3×1 ml). The filters were counted in 3 ml of clearsol I (Nacalai Tesque Inc.). The determination of nonspecific binding was incubated in the presence of 10 μM (−)-nicotine.

The analyses of the experimental results were conducted by using the Accufit Competition Program (Beckman Ltd.).

Biological Experiment 2:

Binding Assays at α1β1γδ Subtype of Nicotinic Acetylcholine Receptors

The affinity of the compounds of the present invention to α1β1γδ subtype of nicotinic acetylcholine receptors was measured by the following method, which was modified method described by Garcha H. S., Thomas P., Spivak C. E., Wonnacott S. & Stolerman I. P., Psychropharmacology, 110, 347-354 (1993).

(1) Preparation of Rat Skeletal Muscles Containing α1β1γδ Subtype of Nicotinic Acetylcholine Receptors

The substantially same animals described in the Biological Experiment 1 were used.

The isolation of α1β1γδ subtype of nicotinic acetylcholine receptors was performed as follow. That is, rat posterior skeletal muscles were isolated just after sacrificed by decapitation, washed with ice-cooled saline solution and then frozen at −80° C. with liquid nitrogen and stored till using. After thawing the frozen muscles, tissue was homogenized (40t w/v) with buffer solution [2.5 mM of sodium phosphate buffer (pH:7.2), 90 mM of NaCl, 2 mM of KCl, 1 mM of EDTA, 2 mM of benzamidine, 0.1 mM of benzethonium chloride, 0.1 mM of PMSF, 0.01% of sodium azide] in Waring blender (Waring blender 34BL97; WARING PRODUCTS DIVISION DYNAMICS CORPORATION OF AMERICA) for 60 seconds. The homogenate were centrifuged under 20,000×G for 60 minutes at 4° C. The supernatant was separated and the resulting pellet was added to the same buffer (1.5 ml/g wet weight), and homogenized under the same conditions. Triton X100 (2% w/v) was added and the mixture was stirred for 3 hours at 4° C. The centrifugation at 100,000×G for 60 minutes at 4° C. yielded the rat muscle extract as supernatant. This was stored at 4° C. for up to 4 weeks, and used for binding assays at receptors.

(2) Experiments of α1β1γδ Subtype of Nicotinic Acetylcholine Receptors Binding

Receptors binding experiments were performed as follow. That is, the extract of rat muscle containing 600-900 μg of protein was added to test tubes containing test compounds and incubated for 15 minutes at 37° C. Then, to this mixture was added 1 nM of [³H]-α-bungarotoxin (α-Bgt) and further incubated for 2 hours. The samples were isolated by vacuum filtration onto Whatman GF/B filters, which were prerinsed with 0.5% polyethylenimine just prior to sample filtration, using Brandel multi manifold cell harvester. The filters were rapidly rinsed with washing solution (10 mM of KH₂PO₄, 150 mM of NaCl, pH 7.2, room temperature) (5×1 ml). The filters were counted in 3 ml of clearsol I (Nacalai Tesque Inc.). The determination of nonspecific binding was incubated in the presence of 1 μM α-Bgt. The solutions containing α-Bgt (labeled/non-labeled) were prepared by using buffer solution containing 0.25% of BSA. In the receptor binding experiments, said buffer solution was added for adjusting the final concentration of BSA to be 0.05%.

The analyses of the experimental results were conducted by the same way as described in the Biological Experiment 1.

Table 15 shows the results of receptor binding studies of the compounds of the present invention and (−)-nicotine as reference compound.

TABLE 15

Affinities for receptors Ki

| | | |
|---|---|---|
| Compound No. | α4β2 | α1β1γδ^{*1} |

| | | | |
|---|---|---|---|
| 2 | 13 | nM | (34%, 6%) |
| 3 | 45 | nM | (34%, 5%) |
| 4 | 67 | nM | (46%, 16%) |
| 7 | 86 | nM | (80%, 51%) |
| 8 | 29 | nM | 395 μM |
| 9 | 7.7 | nM | (43%, 16%) |
| 10 | 1 | nM | (40%, 17%) |
| 11 | 115 | nM | (74%, 53%) |
| 12 | 268 | nM | (79%, 42%) |
| 15 | 950 | nM | n.d. |
| 16 | 392 | nM | (63%, 30%) |
| 18 | 86 | nM | (62%, 18%) |
| 19 | 144 | nM | (69%, 29%) |
| 22 | 429 | nM | (23%, −4%) |
| 25 | 338 | nM | (41%, 7%) |
| 27 | 2 | nM | 45 μM |
| 32 | 580 | nM | (69%, 35%) |
| 33 | 365 | nM | n.d. |
| 36 | 124 | nM | (81%, 34%) |
| 43 | 167 | nM | (71%, 28%) |
| 48 | 82 | nM | 257 μM |
| 49 | 211 | nM | 773 μM |
| 52 | 1.2 | nM | 23 μM |
| 53 | 10 | nM | 83 μM |
| 54 | 108 | nM | 1739 μM |
| 57 | 12 | nM | 86 μM |
| 58 | 6.9 | nM | 32 μM |
| 62 | 70 | nM | 639 μM |
| 64 | 8.1 | nM | 23 μM |
| 65 | 53 | nM | 524 μM |
| 66 | 90 | nM | 841 μM |
| 68 | 203 | nM | 231 μM |
| Nicotine | 1.6 | nM | 182 μM |

^{*1}Values indicated in a parenthesis show control % of [³H]-α-Bgt binding at 100 μM and 1,000 μM of test compounds.
n.d.: not determined.

Biological Experiment 3:

Agonist Activities at Human α4β2 Subtype of Nicotinic Acetylcholine Receptors

The agonist activities of the compounds of the present invention at human α4β2 subtype of nicotinic acetylcholine receptors was evaluated by the following method, which was modified method described by Papke R. L., Thinschmidt J. S., Moulton B. A., Meyer E. M. & Poirier A., Br. J. Pharmacol., 120, 429-438 (1997).

(1) Preparation of cRNA of Human α4β2 Subtype of Nicotinic Acetylcholine Receptors

The cloning of human nicotinic acetylcholine receptor (hnACh-R) α4 cDNA and hnAC-R β2 cDNA were performed, in accordance with the conventional manners, by synthesizing the each DNA primers corresponding to the sequences of hnACh-R α4 cDNA and hnACh-R β2 cDNA [Monteggia L. M. et al., Gene, 155, 189-193 (1995); and Anand R., & Lindstrom J., Nucl. Acids Res., 18, 4272 (1990)], and obtained hnACh-R α4 cDNA and hnACh-R β2 cDNA by polymerase chain reaction (PCR), respectively. The obtained hnACh-R α4 cDNA and hnACh-R β2 cDNA were inserted to the cRNA expression vector (pSP64 polyA) having SP6 RNA promoter to construct hnACh-R α4/pSP64 polyA and hnACh-R β2/pSP64 polyA, respectively. After cutting from expression vector by restriction enzyme (EcoRI), transcription was performed by affecting SP6 RNA polymerase in the presence of cap analogues to obtain hnACh-R α4 cRNA and hnACh-R β2 cRNA, respectively.

(2) Expression of Human α4β2 Subtype Nicotinic Acetylcholine Receptors in Xenopus Oocytes

Oocytes were purchased from Kitanihonseibutsukyohzai Co., Ltd., which were already enucleated from Xenopus laevis, and used in this experiment.

The oocytes were treated with collagenase (Sigma type I; 1 mg/ml) in calcium-free modified Birth's solution (88 mM of NaCl, 1 mM of KCl, 2.4 mM of NaHCO₃, 0.82 mM of MgSO₄, 15 mM of HEPES, pH 7.6) under gently stirring at room temperature for 90 minutes, and washed out the enzyme from the tissue. Then, oocytes were separated from ovarian follicle by tweezers, and isolated oocytes were placed in antibiotics containing modified Birth's solution (88 mM of NaCl, 1 mM of KCl, 2.4 mM of NaHCO₃, 0.82 mM of MgSO₄, 15 mM of HEPES, pH 7.6, and 0.1 v/v % of mixture solution containing of penicillin and streptomycin for incubation; Sigma Co.). Thus treated oocytes were injected with 50 nl of adjusted cRNAs (1.0 mg/ml), that is, each 50 ng of hnACh-R α4 cRNA and hnACh-R β2 cRNA per 1 oocyte by using automatic injector (NANOJECT; DRUMMOND SCIENTIFIC CO.), and further incubated for 4-14 days at 19° C. In oocytes, heterogeneous quintuple [(α4)₂(β2)₃] was composed by translation of injected cRNAs, and ion channel receptors were constructed on cell membrane.

(3) Agonist Activities at Human α4β2 Subtype of Nicotinic Acetylcholine Receptors

The recordings of responses at human α4β2 subtype of nicotinic acetylcholine receptors by means of membrane potential holding method were performed as follow. That is, oocytes were placed in recording chamber with a total volume of 50 μl and were perfused with Ringer's solution (115 mM of NaCl, 2.5 mM of KCl, 1.8 mM of CaCl₂, 10 mM of HEPES, pH 7.3) containing atropine (1 μM) under flow rate of 1 ml/min. The membrane electric potentials were held at −50 mV by mean of the two electric membranes potential holding method (CEZ-1250; Nihon Kohden Co.). Test compounds were added to the perfusion solution, and recorded the peak strength of induced inward current. In order to normalize the responses of test compounds, the responses with acetylcholine (Ach) were recorded before and after application of the test compounds. Generally in the oocytes just after isolated, the response of intrinsic muscarinic acetylcholine receptors, which is inward current caused by activation of calcium dependence chloride ion channels with increase of the intracellular calcium concentration by stimulation of receptors, is observed. However, the complete disappearances of these responses were confirmed when treated with collagenase or added 1 μM of atropine. Furthermore, the oocytes without injection of cRNAs showed no responses by Ach after treatment with collagenase. Therefore, the responses observed in oocytes with injection of hnACh-R α4 cRNA and hnACh-R β2 cRNA, i.e., the inward current induced by the intracellular influx of sodium ion according to the stimulation of receptors, would be the freshly observed responses of human α4β2 subtype nicotinic acetylcholine receptors.

Table 16 shows the results of the agonist activity test of the compounds in the present invention and (−)-nicotine as reference compound.

TABLE 16

| Compound No. | Agonist activity (ED50)^{*1} |
|---|---|
| 2 | 3.4 μM |
| 3 | 43.8 μM |
| 22 | (13.2%) |
| 27 | (18.0%) |
| 45 | (12.0%) |
| 57 | (9.1%) |
| 58 | (27.9%) |
| 62 | (9.6%) |
| nicotine | 11.4 μM |

^{*1}These date are calculated in comparison with the reaction with 10 μM of acetylcholine (100%). Values indicated in a parenthesis show control % by response at 100 μM of the test compounds.

The following are Formulation Examples of the compounds (I) or pharmaceutically acceptable salt thereof according to the present invention

Formulation Example 1 (Tablets):

| | | |
|---|---|---|
| Compound 2 (Fumarate) | 25 | g |
| Lactose | 130 | g |
| Crystalline cellulose | 20 | g |
| Corn starch | 20 | g |
| 3% aqueous solution of hydroxypropylmethyl- | 100 | ml |
| cellulose | | |
| Magnesium stearate | 2 | g |

Fumarate of Compound 2, lactose, crystalline cellulose and corn starch were screened through a 60-mesh sieve, homogenized and charged into a kneader. A 3% aqueous solution of hydroxypropylmethylcellulose was added to the homogeneous mixture and the mixture was further kneaded. The product was granulated by a 16-mesh sieve, dried in air at 50° C., and again granulated by a 16-mesh sieve. Magnesium stearate was added to the granule and mixed again. The mixture was tabletted to produce tablets weighing 200 mg each and having an 8 mm diameter.

Formulation Example 2 (Capsules):

| | | |
|---|---|---|
| Compound 3 (Fumarate) | 25.0 | g |
| Lactose | 125.0 | g |
| Corn starch | 48.5 | g |
| Magnesium stearate | 1.5 | g |

The above components were finely pulverized and thoroughly mixed to produce a homogeneous mixture. The mixture was filled in gelatin capsules, 200 mg per capsule, to obtain capsules.

Formulation Example 3 (Injection):

The fumarate of Compound 58 was filled in an amount of 250 mg in a vial and mixed in situ with approximately 4-5 ml of injectable distilled water to make an injectable solution.

Industrial Applicability

As described above, the compounds of the present invention possess high affinity for α4β2 nicotinic acetylcholine receptor of central nervous systems and activate said α4β2 nicotinic acetylcholine receptor as agonists or modulators. Therefore, the compounds of the present invention are useful for preventing or treating various kinds of diseases, which may be prevented or cured by activating nicotinic acetylcholine receptors.

Especially, the activators for α4β2 nicotinic acetylcholine receptors of the present invention are useful for preventing or treating various diseases such as dementia, senile dementia, presenile dementia, Alzheimer's disease, Parkinson's disease, cerebrovascular dementia, AIDS-related dementia, dementia in Down's syndrome, Tourette's syndrome, neurosis during the chronic cerebral infarction stage, cerebral dysfunction caused by cerebral injury, anxiety, schizophrenia, depression, Huntington's disease, pain and so on.

1. Cyclic amidine compounds represented by the following formula (I): 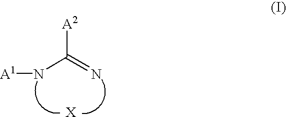
wherein:
A¹ and A² are hydrogen atom, optionally substituted alkyl group; optionally substituted aryl group; or optionally substituted heterocyclic group; and
X is −C(R¹,R²)—C(R³,R⁴)—, —C(R⁵)═C(R⁶)—, —C(R⁷,R⁸)—C(R⁹,R¹⁰)—C(R¹¹,R¹²)—, or —C(R¹³,R¹⁴)—C(R¹⁵,R¹⁶)—NH— (wherein, R¹, R², R³, R⁴, R⁵, R⁶, R⁷, R⁸, R⁹, R¹⁰, R¹¹, R¹², R¹³, R¹⁴, R¹⁵ and R¹⁶ are hydrogen atom; halogen atom; optionally substituted alkyl group; optionally substituted aryl group; or optionally substituted heterocyclic group;
or pharmaceutically acceptable salts thereof.

2. The following compounds represented by the formula (I) of claim 1;
2-(6-chloro-3-pyridyl)-2-imidazoline;
2-(6-chloro-3-pyridyl)-1,4,5,6-tetrahydropyrimidine;
2-(6-chloro-3-pyridyl)-1-methyl-2-imidazoline;
2-(6-chloro-3-pyridyl)-1-methyl-1,4,5,6-tetrahydropyrimidine;
1-(6-chloro-3-pyridyl)methylimidazole;
2-(6-chloro-3-pyridyl)imidazole;
2-(6-chloro-3-pyridyl)methyl-2-imidazoline;
2-(6-chloro-3-pyridyl)methyl-1,4,5,6-tetrahydropyrimidine;
2-(6-chloro-3-pyridyl)methyl-1-methyl-2-imidazoline;
2-(6-chloro-3-pyridyl)methyl-1-methyl-1,4,5,6-tetrahydropyrimidine;
1-(6-chloro-3-pyridyl)methyl-2-methyl-2-imidazoline;
1-(6-chloro-3-pyridyl)methyl-4,4-dimethyl-2-imidazoline;
2-(tetrahydrofuran-3-yl)-1,4,5,6-tetrahydropyrimidine;
2-(tetrahydrofuran-3-yl)-2-imidazoline;
2-(tetrahydrofuran-3-yl)methyl-1,4,5,6-tetrahydropyrimidine;
2-(5-bromo-3-pyridyl)methyl-1,4,5,6-tetrahydropyrimidine;
2-(5-bromo-3-pyridyl)methyl-2-imidazoline;
2-(3-pyridyl)methyl-1,4,5,6-tetrahydropyrimidine;
2-(3-pyridyl)methyl-2-imidazoline;
2-(3-aminophenyl)-1,4,5,6-tetrahydropyrimidine;
2-(3-quinolyl)methyl-1,4,5,6-tetrahydropyrimidine;
2-(2-chloro-5-thiazolyl)-1,4,5,6-tetrahydropyrimidine;
2-(3-quinolyl)methyl-2-imidazoline;
2-(2-chloro-5-thiazolyl)-2-imidazoline;
2-(3-quinolyl)-1,4,5,6-tetrahydropyrimidine;
2-(3-furanyl)methyl-2-imidazoline;
1-(6-chloro-3-pyridyl)methyl-1,4,5,6-tetrahydropyrimidine;
2-(3,5-dimethyl-4-isoxazolyl)methyl-1,4,5,6-tetrahydropyrimidine;
2-(3,5-dimethyl-4-isoxazolyl)methyl-2-imidazoline;
2-(3-thienyl)methyl-1,4,5,6-tetrahydropyrimidine;
2-(3-thienyl)methyl-2-imidazoline;
2-methyl-5-(3-pyridyl)-2-imidazoline;
5-(3-pyridyl)-2-imidazoline;
1,2-bis[(6-chloro-3-pyridyl)methyl]-1,4,5,6-tetrahydropyrimidine;
1-(6-chloro-3-pyridyl)methyl-2-(3-pyridyl)-2-imidazoline;
2-(5,6-dichloro-3-pyridyl)methyl-1,4,5,6-tetrahydropyrimidine;
2-(6-chloro-3-pyridyl)methyl-5-phenyl-1,4,5,6-tetrahydroprymidine;
2-(4-pyridyl)methyl-1,4,5,6-tetrahydropyrimidine;
2-(2-chloro-3-pyridyl)methyl-1,4,5,6-tetrahydropyrimidine;
2-(2,6-dichloro-3-pyridyl)methyl-1,4,5,6-tetrahydropyrimidine;
2-[2-(6-chloro-3-pyridyl)ethyl]-1,4,5,6-tetrahydropyrimidine;
2-[2-(6-chloro-3-pyridyl)ethyl]-2-imidazoline;
2-(6-methyl-3-pyridyl)methyl-1,4,5,6-tetrahydropyrimidine;
1,2-bis[(6-chloro-3-pyridyl)methyl]-2-imidazoline;
2-(6-methyl-3-pyridyl)methyl-2-imidazoline;
2-(6-ethoxy-3-pyridyl)methyl-1,4,5,6-tetrahydropyrimidine;
2-(6-ethoxy-3-pyridyl)methyl-2-imidazoline;
2-(6-fluoro-3-pyridyl)methyl-1,4,5,6-tetrahydropyrimidine;
2-(5,6-dichloro-3-pyridyl)methyl-2-imidazoline;
2-(6-chloro-3-pyridyl)methyl-5,5-dimethyl-1,4,5,6-tetrahydropyrimidine;
2-(2-pyridyl)methyl-1,4,5,6-tetrahydropyrimidine;
1-(5,6-dichloro-3-pyridyl)methyl-1,4,5,6-tetrahydropyrimidine;
2-(5,6-dichloro-3-pyridyl)methyl-1-methyl-2-imidazoline;
2-(6-chloro-3-pyridyl)methyl-4-methyl-1,4,5,6-tetrahydropyrimidine;
1-[2-(6-chloro-3-pyridyl)ethyl]-1,4,5,6-tetrahydropyrimidine;
1-(3-pyridazinyl)methyl-1,4,5,6-tetrahydropyrimidine;
1-(6-methyl-3-pyridyl)methyl-1,4,5,6-tetrahydropyrimdine;
1-(3-pyridyl)methyl-1,4,5,6-tetrahydropyrimidine;
3-(6-chloro-3-pyridyl)methyl-1,4,5,6-tetrahydro-1,2,4-triazine;
2-[1-(6-chloro-3-pyridyl)ethyl]-1,4,5,6-tetrahydropyrimidine;
1-(2-chloro-5-thiazolyl)methyl-1,4,5,6-tetrahydropyrimidine;
1-[2-(6-chloro-3-pyridyl)ethyl]-2-methyl-2-imidazoline;
1-[2-(6-chloro-3-pyridyl)ethyl]-4,4-dimethyl-2-imidazoline;
2-(2-chloro-5-thiazolyl)methyl-1,4,5,6-tetrahydropyrimidine;
2-(2-chloro-5-thiazolyl)methyl-2-imidazoline;
2-(5-pyrimidyl)methyl-1,4,5,6-tetrahydropyrimidine;
2-(5-pyrimidyl)methyl-2-imidazoline;
2-(5-methyl-3-pyridyl)methyl-1,4,5,6-tetrahydropyrimidine. and pharmaceutically acceptable salt thereof.

3. Activators for α4β2 nicotinic acetylcholine receptors containing the compound or pharmaceutically acceptable salt thereof claimed in claim 1 or 2, as active ingredient.

4. The activators for α4β2 nicotinic acetylcholine receptors according to claim 3, wherein said activators are agonists or modulators at α4β2 nicotinic acetylcholine receptors.

5. A medicament for preventing or treating cerebral circulation diseases comprising the activator for α4β2 nicotinic acetylcholine receptors claimed in claim 3 or 4.

6. A medicament for preventing or treating neurodegenerative disease, dementia, motor ataxia, and neuropathy and mental disease comprising the activator for α4β2 nicotinic acetylcholine receptors claimed in claim 3 or 4.

7. The medicament according to claim 6, wherein said neurodegenerative disease is Alzheimer's disease or Parkinson's disease, said dementia is cerebrovascular dementia, said motor ataxia is Tourette's syndrome, and said neuropathy and mental disease is neurosis during the chronic cerebral infarction stage, anxiety or schizophrenia.

8. A medicament for improving the cerebral metabolism, neurotransmission functional disorder and memory disorder, for protecting brain, or having analgesic effect, which comprises the activator for α4β2 nicotinic acetylcholine receptors claimed in claim 3 or 4.

9. A medicament for preventing or treating inflammatory intestinal diseases comprising the activator for α4β2 nicotinic acetylcholine receptors claimed in claim 3 or 4.

10. The use of the compounds claimed in claim 1 or 2 as the activators for α4β2 nicotinic acetylcholine receptors.

11. The method of preventing or treating cerebral circulation diseases which comprises administering activators for α4β2 nicotinic acetylcholine receptors claimed in claim 3 or 4.

12. The method of preventing or treating neurodegenerative diseases, dementia, motor ataxia, and neuropathy and mental disease which comprises administering activators for α4β2 nicotinic acetylcholine receptors claimed in claim 3 or 4.

13. The method according to claim 12, wherein said neurodegenerative disease is Alzheimer's disease or Parkinson's disease, said dementia is cerebrovascular dementia, said motor ataxia is Tourette's syndrome, and said neuropathy and mental disease is neurosis during the chronic cerebral infarction stage, anxiety or schizophrenia.
